SEQUENCE LISTING (181 sequences)

SEQ ID NO: 1 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 2 - protein, 598 aa, synthetic construct
MAGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 3 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 4 - protein, 594 aa, synthetic construct
GGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 5 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 6 - protein, 501 aa, synthetic construct
NKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 7 - protein, 511 aa, synthetic construct
TLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 8 - protein, 510 aa, synthetic construct
LPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 9 - protein, 509 aa, synthetic construct
PQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 10 - protein, 508 aa, synthetic construct
QRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 11 - protein, 507 aa, synthetic construct
RTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 12 - protein, 506 aa, synthetic construct
TIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 13 - protein, 505 aa, synthetic construct
IRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 14 - protein, 504 aa, synthetic construct
RGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 15 - protein, 503 aa, synthetic construct
GKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 16 - protein, 502 aa, synthetic construct
KNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 17 - protein, 501 aa, synthetic construct; identical to SEQ ID NO: 6

SEQ ID NO: 18 - protein, 500 aa, synthetic construct
KHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 19 - protein, 499 aa, synthetic construct
HCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 20 - protein, 498 aa, synthetic construct
CWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 21 - protein, 497 aa, synthetic construct
WSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 22 - protein, 496 aa, synthetic construct
STSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 23 - protein, 495 aa, synthetic construct
TSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 24 - protein, 494 aa, synthetic construct
SKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 25 - protein, 493 aa, synthetic construct
KSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 26 - protein, 492 aa, synthetic construct
STRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 27 - protein, 491 aa, synthetic construct
TRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 28 - protein, 99 aa, synthetic construct
GGGGSERPYACPVESCDRRFSRSDELTRHIRIHTGQKPFQCRICMRNFSRSDHLTTHIRTHTGEKPFACDICGRKFARSDERKRHTKIHLRQKDGGGGS

SEQ ID NO: 29 - protein, 7 aa, synthetic construct
PKKKRKV

SEQ ID NO: 30 - protein, 278 aa, synthetic construct
SIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRAPEQRDALHLPLSWRVKRPRTSIGGGLPDPGTPTAADLAASSTVMREQDEDPFAGAADDFPAFNEEELAWLMELLPQ

SEQ ID NO: 31 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 32 - protein, 63 aa, synthetic construct
SIVAQLSRPDPALAALTNDHLVALACLGGRPALDAVKKGLPHAPALIKRTNRRIPERTSHRVA

SEQ ID NO: 33 - protein, 136 aa, synthetic construct
VDLRTLGYSQQQQEKIKPKVRSTVAQHHEALVGHGFTHAHIVALSQHPAALGTVAVKYQDMIAALPEATHEAIVGVGKSRSGARALEALLTVAGELRGPPLQLDTGQLLKIAKRGGVTAVEAVHAWRNALTGAPLN

SEQ ID NO: 34 - protein, 135 aa, synthetic construct
VDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLN

SEQ ID NO: 35 - protein, 34 aa, synthetic construct
LTPDQVVAIAXXXGGKQALETVQRLLPVLCQDHG

SEQ ID NO: 36 - protein, 34 aa, synthetic construct
LTPEQVVAIAXXXGGKQALETVQRLLPVLCQAHG

SEQ ID NO: 37 - protein, 34 aa, synthetic construct
LTPDQVVAIAXXXGGKQALETVQRLLPVLCQAHG

SEQ ID NO: 38 - protein, 34 aa, synthetic construct
LTPAQVVAIAXXXGGKQALETVQRLLPVLCQDHG

SEQ ID NO: 39 - protein, 20 aa, synthetic construct
LTPEQVVAIAXXXGGRPALE

SEQ ID NO: 40 - protein, 609 aa, synthetic construct
MAPKKKRKVGGGGSERPYACPVESCDRRFSRSDELTRHIRIHTGQKPFQCRICMRNFSRSDHLTTHIRTHTGEKPFACDICGRKFARSDERKRHTKIHLRQKDGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 41 - skipped

SEQ ID NO: 42 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 43 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTKDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 44 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDKLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 45 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 46 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDKLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 47 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 48 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTKDLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 49 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDKLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 50 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVRTAVRKDNHMSTDKLFRRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 51 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVKTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 52 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTRDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 53 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDRLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 54 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFKRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 55 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 56 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 57 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 58 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 59 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 60 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 61 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSESMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRDKFMDNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 62 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSDSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 63 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSREKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 64 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMENLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 65 - protein, 23 aa, synthetic construct
MDYKDHDGDYKDHDIDYKDDDDK

SEQ ID NO: 66 - protein, 9 aa, synthetic construct
MAPKKKRKV

SEQ ID NO: 67 - DNA, 1264 bp, synthetic construct
  source 1..1264: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 68 - DNA, 105 bp, synthetic construct
  source 1..105: mol_type = other DNA
atggtgagcaagggcgaggagctgttcaccggggtggtgcccatcctggtcgagctggacggcgacgtaaacggccacaagttcagcgtgtccggcgagggcgag

SEQ ID NO: 69 - DNA, 37 bp, synthetic construct
  source 1..37: mol_type = other DNA
gtgagtatggatccctctaaaagcgggcatgacttct

SEQ ID NO: 70 - DNA, 35 bp, synthetic construct
  source 1..35: mol_type = other DNA
ccctagaaagatagtctgcgtaaaattgacgcatg

SEQ ID NO: 71 - DNA, 88 bp, synthetic construct
  source 1..88: mol_type = other DNA
agagtagtccagggtttccgagggtttccgtcgactcagctcgtctcgagggcgtactaactgggccctttcccttttttttcctcag

SEQ ID NO: 72 - DNA, 612 bp, synthetic construct
  source 1..612: mol_type = other DNA
ggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 73 - DNA, 49 bp, synthetic construct
  source 1..49: mol_type = other DNA
aataaaatatctttattttcattacatctgtgtgttggttttttgtgtg

SEQ ID NO: 74 - DNA, 63 bp, synthetic construct
  source 1..63: mol_type = other DNA
ccctagaaagataatcatattgtgacgtacgttaaagataatcatgcgtaaaattgacgcatg

SEQ ID NO: 75 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 76 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 77 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 78 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 79 - protein, 73 aa, synthetic construct
SIVAQLSRPDPALAALTNDHLVALACLGGRPALDAVKKGLPHAPALIKRTNRRIPERTSHRVADHAQVVRVLG

SEQ ID NO: 80 - DNA, 4401 bp, synthetic construct
  source 1..4401: mol_type = other DNA
ctagcggtctctttaaccctagaaagatagtctgcgtaaaattgacgcatgcattcttgaaatattgctctctctttctaaatgacgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcggcctccaaggcctgcaggagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggtcggccgccacgaccggtgccaccatgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgctagccttggctccggcgagggcaggggaagtcttctaacatgcggggacgtggaggaaaatcccggcccactcggctacccctacgacgtgcccgactacgccagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaactagatgctgtcgaccgatgcccttgagagccttcaacccagtcagctccttccggtgggcgcggggcaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgactatcgtcgccgcacttattaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaagagacctgtacatacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaagg

SEQ ID NO: 81 - DNA, 52 bp, synthetic construct
  source 1..52: mol_type = other DNA
tggagaccccagaacagccgtaatttaaaggtacacttagtatattactaga

SEQ ID NO: 82 - DNA, 51 bp, synthetic construct
  source 1..51: mol_type = other DNA
tgaagaaacagtgaaatataaaattaagcgagcctcacagacatgtaggaa

SEQ ID NO: 83 - DNA, 52 bp, synthetic construct
  source 1..52: mol_type = other DNA
tgaaacaaaatgatagcaagatatttaacttcagcatatgtagaggtaagaa

SEQ ID NO: 84 - DNA, 51 bp, synthetic construct
  source 1..51: mol_type = other DNA
tttaaaaaaaatagtaataatctattaagtatgagagatgtgcagagagga

SEQ ID NO: 85 - DNA, 52 bp, synthetic construct
  source 1..52: mol_type = other DNA
gtatccgcagagtgagtcctaaatttaaaatctgactacatgtagaaaagcg

SEQ ID NO: 86 - DNA, 52 bp, synthetic construct
  source 1..52: mol_type = other DNA
agtatgataactgatttcaaatatttaaaaaaacaacatgcaagaaagcaga

SEQ ID NO: 87 - DNA, 52 bp, synthetic construct
  source 1..52: mol_type = other DNA
cgtttgctactttagattttttatttaaaaaaaatagtaataatctattaag

SEQ ID NO: 88 - DNA, 52 bp, synthetic construct
  source 1..52: mol_type = other DNA
gaagggagtgatttctcagtattcttaaacctctttctgatgacacttgtac

SEQ ID NO: 89 - DNA, 11 bp, synthetic construct
  source 1..11: mol_type = other DNA
tggagacccca

SEQ ID NO: 90 - DNA, 11 bp, synthetic construct
  source 1..11: mol_type = other DNA
tctagtaatat

SEQ ID NO: 91 - DNA, 11 bp, synthetic construct
  source 1..11: mol_type = other DNA
tgaagaaacag

SEQ ID NO: 92 - DNA, 11 bp, synthetic construct
  source 1..11: mol_type = other DNA
ttcctacatgt

SEQ ID NO: 93 - DNA, 11 bp, synthetic construct
  source 1..11: mol_type = other DNA
tcctacatgtc

SEQ ID NO: 94 - DNA, 11 bp, synthetic construct
  source 1..11: mol_type = other DNA
tgaaacaaaat

SEQ ID NO: 95 - DNA, 11 bp, synthetic construct
  source 1..11: mol_type = other DNA
tcttacctcta

SEQ ID NO: 96 - DNA, 11 bp, synthetic construct
  source 1..11: mol_type = other DNA
ttcttacctct

SEQ ID NO: 97 - DNA, 11 bp, synthetic construct
  source 1..11: mol_type = other DNA
ttaaaaaaaat

SEQ ID NO: 98 - DNA, 11 bp, synthetic construct
  source 1..11: mol_type = other DNA
tttaaaaaaaa

SEQ ID NO: 99 - DNA, 11 bp, synthetic construct
  source 1..11: mol_type = other DNA
tcctctctgca

SEQ ID NO: 100 - DNA, 11 bp, synthetic construct
  source 1..11: mol_type = other DNA
gtatccgcaga

SEQ ID NO: 101 - DNA, 11 bp, synthetic construct
  source 1..11: mol_type = other DNA
cgcttttctac

SEQ ID NO: 102 - DNA, 11 bp, synthetic construct
  source 1..11: mol_type = other DNA
tatccgcagag

SEQ ID NO: 103 - DNA, 11 bp, synthetic construct
  source 1..11: mol_type = other DNA
gcttttctaca

SEQ ID NO: 104 - DNA, 11 bp, synthetic construct
  source 1..11: mol_type = other DNA
agtatgataac

SEQ ID NO: 105 - DNA, 11 bp, synthetic construct
  source 1..11: mol_type = other DNA
tctgctttctt

SEQ ID NO: 106 - DNA, 11 bp, synthetic construct
  source 1..11: mol_type = other DNA
gtatgataact

SEQ ID NO: 107 - DNA, 11 bp, synthetic construct
  source 1..11: mol_type = other DNA
ctgctttcttg

SEQ ID NO: 108 - DNA, 11 bp, synthetic construct
  source 1..11: mol_type = other DNA
cgtttgctact

SEQ ID NO: 109 - DNA, 11 bp, synthetic construct
  source 1..11: mol_type = other DNA
cttaatagatt

SEQ ID NO: 110 - DNA, 11 bp, synthetic construct
  source 1..11: mol_type = other DNA
gtttgctactt

SEQ ID NO: 111 - DNA, 11 bp, synthetic construct
  source 1..11: mol_type = other DNA
ttaatagatta

SEQ ID NO: 112 - DNA, 11 bp, synthetic construct
  source 1..11: mol_type = other DNA
gaagggagtga

SEQ ID NO: 113 - DNA, 11 bp, synthetic construct
  source 1..11: mol_type = other DNA
gtacaagtgtc

SEQ ID NO: 114 - DNA, 11 bp, synthetic construct
  source 1..11: mol_type = other DNA
aagggagtgat

SEQ ID NO: 115 - DNA, 11 bp, synthetic construct
  source 1..11: mol_type = other DNA
tacaagtgtca

SEQ ID NO: 116 - protein, 326 aa, synthetic construct
LTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGRPALE

SEQ ID NO: 117 - protein, 326 aa, synthetic construct
LTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGRPALE

SEQ ID NO: 118 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGRPALE

SEQ ID NO: 119 - protein, 326 aa, synthetic construct
LTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALE

SEQ ID NO: 120 - protein, 326 aa, synthetic construct
LTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASHDGGRPALE

SEQ ID NO: 121 - protein, 326 aa, synthetic construct
LTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGRPALE

SEQ ID NO: 122 - protein, 326 aa, synthetic construct
LTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNIGGRPALE

SEQ ID NO: 123 - protein, 326 aa, synthetic construct
LTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALE

SEQ ID NO: 124 - protein, 326 aa, synthetic construct
LTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGRPALE

SEQ ID NO: 125 - protein, 326 aa, synthetic construct
LTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGRPALE

SEQ ID NO: 126 - protein, 326 aa, synthetic construct
LTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNIGGRPALE

SEQ ID NO: 127 - protein, 326 aa, synthetic construct
TPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALES

SEQ ID NO: 128 - protein, 326 aa, synthetic construct
TPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGRPALES

SEQ ID NO: 129 - protein, 326 aa, synthetic construct
LTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALE

SEQ ID NO: 130 - protein, 326 aa, synthetic construct
TPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALES

SEQ ID NO: 131 - protein, 326 aa, synthetic construct
TPDQVVAIANNNGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALES

SEQ ID NO: 132 - protein, 326 aa, synthetic construct
LTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALE

SEQ ID NO: 133 - protein, 326 aa, synthetic construct
TPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGRPALES

SEQ ID NO: 134 - protein, 326 aa, synthetic construct
TPDQVVAIASNGGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALES

SEQ ID NO: 135 - protein, 326 aa, synthetic construct
TPDQVVAIANNNGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGRPALES

SEQ ID NO: 136 - protein, 326 aa, synthetic construct
TPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALES

SEQ ID NO: 137 - protein, 326 aa, synthetic construct
TPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALES

SEQ ID NO: 138 - protein, 326 aa, synthetic construct
LTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGRPALE

SEQ ID NO: 139 - protein, 326 aa, synthetic construct
TPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNIGGRPALES

SEQ ID NO: 140 - protein, 326 aa, synthetic construct
TPDQVVAIASNGGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASHDGGRPALES

SEQ ID NO: 141 - protein, 326 aa, synthetic construct
TPDQVVAIASNIGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALES

SEQ ID NO: 142 - protein, 326 aa, synthetic construct
LTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALE

SEQ ID NO: 143 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 144 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 145 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 146 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 147 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 148 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 149 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 150 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 151 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 152 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 153 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 154 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 155 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 156 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 157 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 158 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 159 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 160 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 161 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 162 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 163 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 164 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 165 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 166 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIANNNGGKQALETVQRLLPVLCQDHGLTPEQVVAIASNIGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIANNNGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 167 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASNIGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 168 - protein, 1088 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKYHGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIANNNGGKQALETVQRLLPVLCQAHGLTPDQVVAIANNNGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 169 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNIGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 170 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIANNNGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASHDGGKQALETVQRLLPVLCQDHGLTPDQVVAIASHDGGKQALETVQRLLPVLCQDHGLTPEQVVAIASNIGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 171 - protein, 1089 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASNGGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNIGGKQALETVQRLLPVLCQAHGLTPDQVVAIASNIGGKQALETVQRLLPVLCQAHGLTPAQVVAIASNGGGKQALETVQRLLPVLCQDHGLTPDQVVAIASNIGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVADHAQVVRVLGGGGGSPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 172 - DNA, 50 bp, synthetic construct
  source 1..50: mol_type = other DNA
tgcagatgaacgcaatctccggcttaactactgactgaagttcatctgca

SEQ ID NO: 173 - DNA, 50 bp, synthetic construct
  source 1..50: mol_type = other DNA
tgcagatgaacgcaatctccggtttaaatactgactgaagttcatctgca

SEQ ID NO: 174 - DNA, 50 bp, synthetic construct
  source 1..50: mol_type = other DNA
tgcagatgaacgcaatctccggcttaaatactgactgaagttcatctgca

SEQ ID NO: 175 - DNA, 52 bp, synthetic construct
  source 1..52: mol_type = other DNA
tgcagatgaacgcaatctcacggcttaactactgacctgaagttcatctgca

SEQ ID NO: 176 - DNA, 52 bp, synthetic construct
  source 1..52: mol_type = other DNA
tgcagatgaacgcaatctcacggtttaaatactgacctgaagttcatctgca

SEQ ID NO: 177 - DNA, 52 bp, synthetic construct
  source 1..52: mol_type = other DNA
tgcagatgaacgcaatctcacggcttaaatactgacctgaagttcatctgca

SEQ ID NO: 178 - DNA, 52 bp, synthetic construct
  source 1..52: mol_type = other DNA
tgcagatgaacgcaatctcacggtttaagtactgacctgaagttcatctgca

SEQ ID NO: 179 - DNA, 52 bp, synthetic construct
  source 1..52: mol_type = other DNA
tgcagatgaacgcaatctcacggcttaagtactgacctgaagttcatctgca

SEQ ID NO: 180 - protein, 609 aa, synthetic construct; identical to SEQ ID NO: 40

SEQ ID NO: 181 - protein, 5 aa, synthetic construct
GGGGS

(19) United States
(12) Patent Application Publication
(10) Pub. No.: US 2026/0022354 A1
(43) Pub. Date: Jan. 22, 2026

(54) TRANSPOSASES AND USES THEREOF

(71) Applicant: Poseida Therapeutics, Inc., San Diego, CA (US)

(72) Inventors: Blair B. MADISON, San Diego, CA (US); Joseph S. LUCAS, San Diego, CA (US); J. Andres VALDERRAMA, San Diego, CA (US); Brandon Rios MOLINA, San Diego, CA (US); Olga BATALOV, San Diego, CA (US)

(21) Appl. No.: 19/349,745

(22) Filed: Oct. 3, 2025

Related U.S. Application Data
(63) Continuation of application No. PCT/US2024/022988, filed on Apr. 4, 2024.
(60) Provisional application No. 63/494,306, filed on Apr. 5, 2023.

(51) Int. Cl. C12N 9/12; C12N 5/10

(52) U.S. Cl. CPC C12N 9/1241; C12N 5/10; C07K 2319/60; C07K 2319/705; C07K 2319/81

(57) ABSTRACT

Provided herein are fusion proteins comprising transposase domains and DNA targeting domains. In particular, the DNA targeting domains may be targeted to the lipoprotein A (LPA) gene. Also provided are methods of making the transposase domains and fusion proteins, cells that are modified using the fusion proteins provided herein and methods of treatment using such cells.

### CROSS-REFERENCE TO RELATED APPLICATIONS

The present application is a continuation of International Patent Application No. PCT/US2024/022988, filed Apr. 4, 2024, which claims the benefit of U.S. Provisional Patent Application No. 63/494,306 filed Apr. 5, 2023, each of which is incorporated herein by reference in its entirety.

### REFERENCE TO SEQUENCE LISTING SUBMITTED ELECTRONICALLY

The instant application contains a Sequence Listing, which has been submitted electronically in XML file format, and is herein incorporated by reference into the specification in its entirety. The XML file containing the Sequence Listing XML is named “000218-0143-101-SL.xml,” was created on Oct. 3, 2025 and is 237,138 bytes in size.

### FIELD

This disclosure generally relates to fusion proteins comprising transposase domains and DNA targeting domains. Also provided are methods of use of the fusion proteins for site-specific transposition.

### BACKGROUND

Transposases may be used to introduce non-endogenous DNA sequences into genomic DNA, and are in many ways advantageous to other methods gene editing. However, there remains an unmet need for site-specific transposases for use in e.g., gene editing.

The lipoprotein (a), or LPA, gene evolved from a duplication event of the neighboring plasminogen (PLG) gene. This duplication event occurred during primate evolution about 40 million years ago. Both genes contain looped structures known as kringle domains. In LPA, the kringle domains have segmentally duplicated such that each copy of LPA can contain up to 50 copies of the kringle domains (Schmidt et al., J Lipid Res. 2016 August; 57(8):1339-59). At the genomic DNA level, each kringle domain repeat spans about 5.5 kb of DNA, each consisting of two exons and two introns. The intronic portion of the repeats contain several potential target sites for site-specific transposase-mediated integration.

LPA is an attractive target for site-specific transposition because it contains multiple copies of the same target site, increasing the chance of integrating a transposon in at least one. Furthermore, LPA is highly expressed in hepatocytes meaning it likely has an open chromosomal landscape amenable to editing and supporting high expression of integrated transgenes. It is a non-essential gene and knockout is associated with lower cholesterol levels. Combined, these traits make it a potential target site for gene therapies.

### SUMMARY

In one aspect, provided herein is a fusion protein comprising a DNA targeting domain and a transposase domain comprising the sequence set forth in SEQ ID NO: 4, wherein the DNA targeting domain binds to a nucleic acid sequence encoding an LPA repeat element. In some embodiments, the DNA targeting domain comprises one, two or three Zinc Finger Motifs. In some embodiments, the DNA targeting domain comprises one or more TAL domains. In some embodiments, the TAL domain comprises the sequence set forth in any one of SEQ ID NOs: 35-38. In some embodiments, the DNA targeting domain binds to a nucleic acid sequence encoding a kringle domain repeat element or an intron adjacent to a sequence encoding a kringle domain repeat element in the LPA gene.

In some embodiments, the transposase domain and the DNA targeting domain are connected by a linker. In some embodiments, the linker comprises the sequence GGGGS (SEQ ID NO: 181).

In some embodiments, the DNA targeting domain is inserted into the N-terminus of the transposase domain at a position after the 82^{nd} amino acid and before the 105^{th} amino acid of SEQ ID NO:4. In some embodiments, the DNA targeting domain replaces one or more amino acid(s) in the transposase domain between, and including, the 83^{rd} amino acid and the 105^{th} amino acid of SEQ ID NO: 4. In some embodiments, the transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

In some embodiments, the transposase domain comprises the sequence set forth in any one of SEQ ID NOs: 7-27. In some embodiments, the transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D.

In another aspect, provided herein is a polynucleotide comprising a nucleic acid sequence encoding a fusion protein described herein.

In another aspect, provided herein is a vector comprising a polynucleotide described herein.

In another aspect, provided herein is a method of integrating a transgene into a genomic target site of a cell, the method comprising introducing into the cell a fusion protein described herein and a transposon, wherein the transposon comprises, in 5′ to 3′ order: a 5′ITR, the transgene, and a 3′ ITR. In some embodiments, the transposon further comprises an exogenous promoter between the 5′ ITR and the transgene. In some embodiments, the transgene encodes a detectable marker. In some embodiments, the detectable marker is GFP. In some embodiments, the transgene is a gene that is (a) not expressed by the cell prior to the introduction of the fusion protein and the transposon or (b) exhibits decreased, insufficient, and/or altered expression by the cell prior to the introduction of the fusion protein and the transposon.

In some embodiments, the genomic target site is located on the LPA gene. In some embodiments, the genomic target site is located in a repetitive element. In some embodiments, the repetitive element is an LPA repeat element. In some embodiments, the genomic target site is located in an intron of a gene. In some embodiments, the genomic target site is located in the intron of the LPA gene. In some embodiments, the cell is in vivo.

In another aspect, provided herein is a method of modifying the genome of a cell, the method comprising: providing the cell with a fusion protein described herein, wherein the cell comprises a modified binding site comprising, in 5′ to 3′ order, the sequence of a target site for the DNA targeting domain, a first spacer, a TTAA target integration site for SPB, a second spacer, and the reverse complement of the sequence of the target site for the DNA targeting domain. In some embodiments, the target integration site comprises the sequence TTAA. In some embodiments, the target integration site comprises the nucleic acid sequence set forth in any one of SEQ ID NOs: 81-88

In another aspect, provided herein is an integration cassette for site-specific transposition of a nucleic acid into the genome of a cell comprising a nucleic acid comprising or consisting of a central transposon ITR integration site TTAA sequence flanked by an upstream TAL array target sequence and a downstream TAL array target sequence, wherein each of the upstream and the downstream TAL array target sequences is separated from the TTAA sequence by 12 or 13 base pairs. In some embodiments, the integration site comprises the sequence TTAA. In some embodiments, the integration site comprises the nucleic acid sequence set forth in any one of SEQ ID NOs: 81-88 In some embodiments, each of the upstream and downstream TAL array target site sequences are the same. In some embodiments, each of the upstream and downstream TAL array target site sequences are different. In some embodiments, each of the upstream and downstream TAL Array target sites target a 7-30 bp sequence of an LPA repeat element.

In another aspect, provided herein is a cell, comprising an integration cassette described herein stably integrated into the genome of the cell.

In another aspect, provided herein is a method for site-specific transposition of a DNA molecule into the genome of a cell, comprising introducing into a cell comprising an integration cassette described herein: a nucleic acid encoding a fusion protein comprising a DNA binding domain and a transposase; wherein the fusion protein is expressed in the cell; and a DNA molecule comprising a transposon; wherein the expressed fusion protein integrates the transposon by site-specific transposition into the TTAA integration site of the stably integrated integration cassette.

In another aspect, provided herein is a method for generating an engineered cell by site-specific transposition, comprising introducing into a cell comprising an integration cassette described herein: a nucleic acid encoding a fusion protein comprising a DNA binding domain and a transposase; wherein the fusion protein is expressed in the cell; and a DNA molecule comprising a transposon; wherein the expressed fusion protein integrates the transposon by site-specific transposition into the TTAA integration site of the stably integrated integration cassette thereby generating the engineered cell. In some embodiments, the sequence TTAA. In some embodiments, the integration site comprises the nucleic acid sequence set forth in any one of SEQ ID NOs: 81-88

### BRIEF DESCRIPTION OF DRAWINGS

FIGS. 1A-1D illustrate the introduction of DNA binding domains into a transposase using obligate heterodimers.

FIG. 2 is a schematic showing the Split GFP Splicing Site Specific Reporter.

FIG. 3 is a schematic showing the catalytic ssSPB dimer bound to an excised transposon and recognizing its genomic integration target site.

### DETAILED DESCRIPTION

Provided herein are fusion proteins comprising transposase domains and DNA targeting domains. In particular, the DNA targeting domains may be targeted to the lipoprotein A (LPA) gene. Also provided are methods of making the transposase domains and fusion proteins, cells that are modified using the fusion proteins provided herein and methods of treatment using such cells.

In some embodiments, provided herein is a fusion protein comprising an SPB or PBx domain and a DNA targeting domain. DNA targeting domains are described further below.

### Transposase Domains

In one aspect, provided herein are fusion proteins comprising one or more transposase domains. In some embodiments, the transposase domain is a piggyBac transposase domain. In some embodiments, the piggyBac transposase domain is a hyperactive piggyBac transposase domain. In preferred embodiments, the transposase domain is a Super piggyBac™ transposase domains (SPB). Non-limiting examples of SPB transposases are described in detail in U.S. Pat. Nos. 6,218,182; 6,962,810; 8,399,643 and PCT Publication No. WO 2010/099296, each of which is incorporated herein by reference in its entirety for examples of transposase domains that may be used in the fusion proteins described herein.

In some embodiments, the transposase domain is a Super PiggyBac transposase (SPB) domain. An SPB comprises one or more hyperactivity mutations compared to the wildtype piggyBac transposase. An illustrative wildtype SPB sequence comprising a nuclear localization sequence (NLS) is shown in SEQ ID NO: 1, with the NLS shown in italics, and hyperactive mutations shown in bold. The numbering of sequence of the SPB transposase domain for the purpose of describing deletions and mutations begins at residue 12 of SEQ ID NO: 1.

(SEQ ID NO: 1)
MAPKKKRKVGGGGSSLDDEHILSALLQSDDELVGEDSDSEVSDHVSEDDVQSD
TEEAFIDEVHEVQPTSSGSEILDEQNVIEQPGSSLASNRILTLPQRTIRGKNKHCWSTSKST
RRSRVSALNIVRSQRGPTRMCRNIYDPLLCFKLFFTDEIISEIVKWTNAEISLKRRESMTSA
TFRDTNEDEIYAFFGILVMTAVRKDNHMSTDDLFDRSLSMVYVSVMSRDRFDFLIRCLR
MDDKSIRPTLRENDVFTPVRKIWDLFIHQCIQNYTPGAHLTIDEQLLGFRGRCPFR VYIPN
KPSKYGIKILMMCDSGTKYMINGMPYLGRGTQTNGVPLGEYYVKELSKPVHGSCRNIT
CDNWFTSIPLAKNLLQEPYKLTIVGTVRSNKREIPEVLKNSRSRPVGTSMFCFDGPLTLVS
YKPKPAKMVYLLSSCDEDASINESTGKPQMVMYYNQTKGGVDTLDQMCSVMTCSRKT
NRWPMALLYGMINIACINSFIIYSHNVSSKGEKVQSRKKFMRNLYMSLTSSFMRKRLEA
PTLKRYLRDNISNILPKEVPGTSDDSTEEPVMKKRTYCTYCPSKIRRKANASCKKCKKVI
CREHNIDMCQSCF.

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 1. In some embodiments, a fusion protein described herein comprises a transposase domain comprising the amino acid sequence set forth in SEQ ID NO: 1 with one, two, three, four or five conservative amino acid substitutions. In some embodiments, a fusion protein described herein comprises a transposase domain comprising the amino acid sequence set forth in SEQ ID NO: 1.

An illustrative sequence of wildtype SPB transposase which is lacking the NLS domain is set forth in SEQ ID NO: 2. The numbering of sequence of the SPB transposase domain for the purpose of describing deletions and mutations begins at residue 5 of SEQ ID NO: 2. In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 2. In some embodiments, a fusion protein described herein comprises a transposase domain comprising the amino acid sequence set forth in SEQ ID NO: 2 with one, two, three, four or five conservative amino acid substitutions. In some embodiments, a fusion protein described herein comprises a transposase domain comprising the amino acid sequence set forth in SEQ ID NO: 2.

The transposase domains used in the fusion proteins described herein can be isolated or derived from an insect, vertebrate, crustacean or urochordate as described in more detail in PCT Publications No. WO 2019/173636 and No. WO 2020/051374. In preferred aspects, the SPB transposase domain is isolated or derived from the insect Trichoplusia ni (GenBank Accession No. AAA87375), Bombyx mori (GenBank Accession No. BAD11135), or Macdunnoughia crassisigna (GenBank Accession No. ABZ85926.1).

In some embodiments, the transposase domain is integration deficient. An integration deficient transposase domain is a transposase that can excise its corresponding transposon, but that integrates the excised transposon at a lower frequency than a corresponding wildtype transposase. Examples of integration deficient transposases are disclosed in U.S. Pat. Nos. 6,218,185; 6,962,810, 8,399,643 and WO 2019/173636, each of which is incorporated herein by reference in its entirety for examples of transposase domains that may be used in the fusion proteins described herein. A list of integration deficient amino acid substitutions is disclosed in U.S. Pat. No. 10,041,077, which is incorporated herein by reference in its entirety for examples of mutations that may be introduced into a transposase domain described herein.

A wildtype SPB may be rendered integration deficient by introducing mutations, for example, K93A, R372A, K375A, R376A and/or D450N (relative to SEQ ID NO: 2, with numbering beginning at residue 5). It is believed that the introduction of mutations R372A, K375A, R376A and D450N renders the transposase integration deficient, but retains the excision function. An illustrative sequence of an integration-deficient transposase domain is PBx comprising an NLS is set forth in SEQ ID NO: 3. In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 3. In some embodiments, a fusion protein described herein comprises a transposase domain comprising the amino acid sequence set forth in SEQ ID NO: 3 with one, two, three, four or five conservative amino acid substitutions. In some embodiments, a fusion protein described herein comprises a transposase domain comprising the amino acid sequence set forth in SEQ ID NO: 3.

The sequence of an integration deficient PBx transpose domain not comprising an NLS is set forth in SEQ ID NO: 4:

(SEQ ID NO: 4)
GGSSLDDEHILSALLQSDDELVGEDSDSEVSDHVSEDDVQSDTEEAFIDEVHEVQPTSSG
SEILDEQNVIEQPGSSLASNRILTLPQRTIRGKNKHCWSTSKSTRRSRVSALNIVRSQRGPT
RMCRNIYDPLLCFKLFFTDEIISEIVKWTNAEISLKRRESMTSATFRDTNEDEIYAFFGILV
MTAVRKDNHMSTDDLFDRSLSMVYVSVMSRDRFDFLIRCLRMDDKSIRPTLRENDVFT
PVRKIWDLFIHQCIQNYTPGAHLTIDEQLLGFRGRCPFRVYIPNKPSKYGIKILMMCDSGT
KYMINGMPYLGRGTQTNGVPLGEYYVKELSKPVHGSCRNITCDNWFTSIPLAKNLLQEP
YKLTIVGTVASNAREIPEVLKNSRSRPVGTSMFCFDGPLTLVSYKPKPAKMVYLLSSCDE
DASINESTGKPQMVMYYNQTKGGVDTLNQMCSVMTCSRKTNRWPMALLYGMINIACI
NSFIIYSHNVSSKGEKVQSRKKFMRNLYMSLTSSFMRKRLEAPTLKRYLRDNISNILPKE
VPGTSDDSTEEPVMKKRTYCTYCPSKIRRKANASCKKCKKVICREHNIDMCQSCF.

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 4. In some embodiments, a fusion protein described herein comprises a transposase domain comprising the amino acid sequence set forth in SEQ ID NO: 4 with one, two, three, four or five conservative amino acid substitutions. In some embodiments, a fusion protein described herein comprises a transposase domain comprising the amino acid sequence set forth in SEQ ID NO: 4.

### Transposase Domains Comprising N-Terminal Deletions

In some embodiments, provided herein are transposase domains (e.g., SPB transposase domains or PBx transposase domains) comprising a deletion of a portion of the amino terminus (also referred to as the “N-terminus” or the “N-terminal Domain,” or “NTD) of the transposase domain. SPB transposase domains or PBx transposase domains comprising N-terminal Domain deletions have been previously described in International Patent Application Publication No. PCT/US2022/77549, which is incorporated herein by reference in its entirety for examples of transposase domains that may be used in the fusion proteins described herein.

Illustrative sequences of an SPB transposase domain with a deletion of amino acids 1-93 of the N-terminus and of a PBx transposase domain with a deletion of amino acids 1-93 of the N-terminus are shown in SEQ ID NOs: 5 and 6, respectively:

(SEQ ID NO: 5)
NKHCWSTSKSTRRSRVSALNIVRSQRGPTRMCRNIYDPLLCFKLFFTDEIISEIV
KWTNAEISLKRRESMTSATFRDTNEDEIYAFFGILVMTAVRKDNHMSTDDLFDRSLSMV
YVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGRGTQTNGVPLGE
YYVKELSKPVHGSCRNITCDNWFTSIPLAKNLLQEPYKLTIVGTVRSNKREIPEVLKNSR
SRPVGTSMFCFDGPLTLVSYKPKPAKMVYLLSSCDEDASINESTGKPQMVMYYNQTKG
GVDTLDQMCSVMTCSRKTNRWPMALLYGMINIACINSFIIYSHNVSSKGEKVQSRKKFM
RNLYMSLTSSFMRKRLEAPTLKRYLRDNISNILPKEVPGTSDDSTEEPVMKKRTYCTYCP
SKIRRKANASCKKCKKVICREHNIDMCQSCF.

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 5. In some embodiments, a fusion protein described herein comprises a transposase domain comprising the amino acid sequence set forth in SEQ ID NO: 5 with one, two, three, four or five conservative amino acid substitutions. In some embodiments, a fusion protein described herein comprises a transposase domain comprising the amino acid sequence set forth in SEQ ID NO: 5.

(SEQ ID NO: 6)
NKHCWSTSKSTRRSRVSALNIVRSQRGPTRMCRNIYDPLLCFKLFFTDEIISEIV
KWTNAEISLKRRESMTSATFRDTNEDEIYAFFGILVMTAVRKDNHMSTDDLFDRSLSMV
YVSVMSRDRFDFLIRCLRMDDKSIRPTLRENDVFTPVRKIWDLFIHQCIQNYTPGAHLTI
DEQLLGFRGRCPFRVYIPNKPSKYGIKILMMCDSGTKYMINGMPYLGRGTQINGVPLGE
YYVKELSKPVHGSCRNITCDNWFTSIPLAKNLLQEPYKLTIVGTVASNAREIPEVLKNSR
SRPVGTSMFCFDGPLTLVSYKPKPAKMVYLLSSCDEDASINESTGKPQMVMYYNQTKG
GVDTLNQMCSVMTCSRKTNRWPMALLYGMINIACINSFIIYSHNVSSKGEKVQSRKKFM
RNLYMSLTSSFMRKRLEAPTLKRYLRDNISNILPKEVPGTSDDSTEEPVMKKRTYCTYCP
SKIRRKANASCKKCKKVICREHNIDMCQSCF.

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 6. In some embodiments, a fusion protein described herein comprises a transposase domain comprising the amino acid sequence set forth in SEQ ID NO: 6 with one, two, three, four or five conservative amino acid substitutions. In some embodiments, a fusion protein described herein comprises a transposase domain comprising the amino acid sequence set forth in SEQ ID NO: 6.

Other illustrative sequences of PBx transposase domains comprising N-terminal deletions are set forth in SEQ ID NOs: 7-27 in Table 1.

In some embodiments, a fusion protein described herein comprises a transposase domain comprising an amino acid sequence that is at least 80%, at least 85%, at least 90%, at least 9100, at least 92%, at least 9300, at least 94%, at least 95%, at least 96%, at least 97%, at least 98%, or at least 99 identical to the amino acid sequence set forth in any one of SEQ ID NOs: 7-27. In some embodiments, a fusion protein described herein comprises a transposase domain comprising the amino acid sequence set forth in any one of SEQ TD NOs: 7-27 with one, two, three, four or five conservative amino acid substitutions. In some embodiments, a fusion protein described herein comprises a transposase domain comprising the amino acid sequence set forth in any one of SEQ ID NOs: 7-27.

TABLE 1
Illustrative sequences of N-terminally deleted PBx Domains

| Deletion | Sequence |
|---|---|
| PBx | TLPQRTIRGKNKHCWSTSKSTRRSRVSALNIVRSQRGPTRMCRNIYDPLL |
| Delta 83 | CFKLFFTDEIISEIVKWTNAEISLKRRESMTSATFRDTNEDEIYAFFGILVM |
| N- | TAVRKDNHMSTDDLFDRSLSMVYVSVMSRDRFDFLIRCLRMDDKSIRPT |
| Terminal | LRENDVFTPVRKIWDLFIHQCIQNYTPGAHLTIDEQLLGFRGRCPFRVYIP |
| | NKPSKYGIKILMMCDSGTKYMINGMPYLGRGTQTNGVPLGEYYVKELS |
| | KPVHGSCRNITCDNWFTSIPLAKNLLQEPYKLTIVGTVASNAREIPEVLK |
| | NSRSRPVGTSMFCFDGPLTLVSYKPKPAKMVYLLSSCDEDASINESTGKP |
| | QMVMYYNQTKGGVDTLNQMCSVMTCSRKTNRWPMALLYGMINIACIN |
| | SFIIYSHNVSSKGEKVQSRKKFMRNLYMSLTSSFMRKRLEAPTLKRYLR |
| | DNISNILPKEVPGTSDDSTEEPVMKKRTYCTYCPSKIRRKANASCKKCKK |
| | VICREHNIDMCQSCF (SEQ ID NO: 7) |
| PBx | LPQRTIRGKNKHCWSTSKSTRRSRVSALNIVRSQRGPTRMCRNIYDPLLC |
| Delta 84 | FKLFFTDEIISEIVKWTNAEISLKRRESMTSATFRDTNEDEIYAFFGILVMT |
| N- | AVRKDNHMSTDDLFDRSLSMVYVSVMSRDRFDFLIRCLRMDDKSIRPTL |
| Terminal | RENDVFTPVRKIWDLFIHQCIQNYTPGAHLTIDEQLLGFRGRCPFRVYIPN |
| | KPSKYGIKILMMCDSGTKYMINGMPYLGRGTQTNGVPLGEYYVKELSK |
| | PVHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCF (SEQ ID NO: 8) |
| PBx | PQRTIRGKNKHCWSTSKSTRRSRVSALNIVRSQRGPTRMCRNIYDPLLCF |
| Delta 85 | KLFFTDEIISEIVKWTNAEISLKRRESMTSATFRDTNEDEIYAFFGILVMTA |
| N- | VRKDNHMSTDDLFDRSLSMVYVSVMSRDRFDFLIRCLRMDDKSIRPTLR |
| Terminal | ENDVFTPVRKIWDLFIHQCIQNYTPGAHLTIDEQLLGFRGRCPFRVYIPNK |
| | PSKYGIKILMMCDSGTKYMINGMPYLGRGTQTNGVPLGEYYVKELSKP |
| | VHGSCRNITCDNWFTSIPLAKNLLQEPYKLTIVGTVASNAREIPEVLKNS |
| | RSRPVGTSMFCFDGPLTLVSYKPKPAKMVYLLSSCDEDASINESTGKPQ |
| | MVMYYNQTKGGVDTLNQMCSVMTCSRKTNRWPMALLYGMINIACINS |
| | FIIYSHNVSSKGEKVQSRKKFMRNLYMSLTSSFMRKRLEAPTLKRYLRD |
| | NISNILPKEVPGTSDDSTEEPVMKKRTYCTYCPSKIRRKANASCKKCKKV |
| | ICREHNIDMCQSCF (SEQ ID NO: 9) |
| PBx | QRTIRGKNKHCWSTSKSTRRSRVSALNIVRSQRGPTRMCRNIYDPLLCFK |
| Delta 86 | LFFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| | SKYGIKILMMCDSGTKYMINGMPYLGRGTQTNGVPLGEYYVKELSKPV |
| | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| | YSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICR |
| | EHNIDMCQSCF (SEQ ID NO: 10). |
| PBx | RTIRGKNKHCWSTSKSTRRSRVSALNIVRSQRGPTRMCRNIYDPLLCFKL |
| Delta 87 | FFTDEIISEIVKWTNAEISLKRRESMTSATFRDTNEDEIYAFFGILVMTAV |
| N- | RKDNHMSTDDLFDRSLSMVYVSVMSRDRFDFLIRCLRMDDKSIRPTLRE |
| Terminal | NDVFTPVRKIWDLFIHQCIQNYTPGAHLTIDEQLLGFRGRCPFRVYIPNKP |
| | SKYGIKILMMCDSGTKYMINGMPYLGRGTQTNGVPLGEYYVKELSKPV |
| | HGSCRNITCDNWFTSIPLAKNLLQEPYKLTIVGTVASNAREIPEVLKNSRS |
| | RPVGTSMFCFDGPLTLVSYKPKPAKMVYLLSSCDEDASINESTGKPQMV |
| | MYYNQTKGGVDTLNQMCSVMTCSRKTNRWPMALLYGMINIACINSFII |
| | YSHNVSSKGEKVQSRKKFMRNLYMSLTSSFMRKRLEAPTLKRYLRDNIS |
| | NILPKEVPGTSDDSTEEPVMKKRTYCTYCPSKIRRKANASCKKCKKVICR |
| | EHNIDMCQSCF (SEQ ID NO: 11) |
| PBx | TIRGKNKHCWSTSKSTRRSRVSALNIVRSQRGPTRMCRNIYDPLLCFKLF |
| Delta 88 | FTDEIISEIVKWTNAEISLKRRESMTSATFRDTNEDEIYAFFGILVMTAVR |
| N- | KDNHMSTDDLFDRSLSMVYVSVMSRDRFDFLIRCLRMDDKSIRPTLREN |
| Terminal | DVFTPVRKIWDLFIHQCIQNYTPGAHLTIDEQLLGFRGRCPFRVYIPNKPS |
| | KYGIKILMMCDSGTKYMINGMPYLGRGTQTNGVPLGEYYVKELSKPVH |
| | GSCRNITCDNWFTSIPLAKNLLQEPYKLTIVGTVASNAREIPEVLKNSRSR |
| | PVGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCF (SEQ ID NO: 12) |
| PBx | IRGKNKHCWSTSKSTRRSRVSALNIVRSQRGPTRMCRNIYDPLLCFKLFF |
| Delta 89 | TDEIISEIVKWTNAEISLKRRESMTSATFRDTNEDEIYAFFGILVMTAVRK |
| N- | DNHMSTDDLFDRSLSMVYVSVMSRDRFDFLIRCLRMDDKSIRPTLREND |
| Terminal | VFTPVRKIWDLFIHQCIQNYTPGAHLTIDEQLLGFRGRCPFRVYIPNKPSK |
| | YGIKILMMCDSGTKYMINGMPYLGRGTQTNGVPLGEYYVKELSKPVHG |
| | SCRNITCDNWFTSIPLAKNLLQEPYKLTIVGTVASNAREIPEVLKNSRSRP |
| | VGTSMFCFDGPLTLVSYKPKPAKMVYLLSSCDEDASINESTGKPQMVM |
| | YYNQTKGGVDTLNQMCSVMTCSRKTNRWPMALLYGMINIACINSFIIYS |
| | HNVSSKGEKVQSRKKFMRNLYMSLTSSFMRKRLEAPTLKRYLRDNISNI |
| | LPKEVPGTSDDSTEEPVMKKRTYCTYCPSKIRRKANASCKKCKKVICRE |
| | HNIDMCQSCF (SEQ ID NO: 13) |
| PBx | RGKNKHCWSTSKSTRRSRVSALNIVRSQRGPTRMCRNIYDPLLCFKLFFT |
| Delta 90 | DEIISEIVKWTNAEISLKRRESMTSATFRDTNEDEIYAFFGILVMTAVRKD |
| N- | NHMSTDDLFDRSLSMVYVSVMSRDRFDFLIRCLRMDDKSIRPTLRENDV |
| Terminal | FTPVRKIWDLFIHQCIQNYTPGAHLTIDEQLLGFRGRCPFRVYIPNKPSKY |
| | GIKILMMCDSGTKYMINGMPYLGRGTQTNGVPLGEYYVKELSKPVHGS |
| | CRNITCDNWFTSIPLAKNLLQEPYKLTIVGTVASNAREIPEVLKNSRSRPV |
| | GTSMFCFDGPLTLVSYKPKPAKMVYLLSSCDEDASINESTGKPQMVMY |
| | YNQTKGGVDTLNQMCSVMTCSRKTNRWPMALLYGMINIACINSFIIYSH |
| | NVSSKGEKVQSRKKFMRNLYMSLTSSFMRKRLEAPTLKRYLRDNISNIL |
| | PKEVPGTSDDSTEEPVMKKRTYCTYCPSKIRRKANASCKKCKKVICREH |
| | NIDMCQSCF (SEQ ID NO: 14) |
| PBx | GKNKHCWSTSKSTRRSRVSALNIVRSQRGPTRMCRNIYDPLLCFKLFFTD |
| Delta 91 | EIISEIVKWTNAEISLKRRESMTSATFRDTNEDEIYAFFGILVMTAVRKDN |
| N- | HMSTDDLFDRSLSMVYVSVMSRDRFDFLIRCLRMDDKSIRPTLRENDVF |
| Terminal | TPVRKIWDLFIHQCIQNYTPGAHLTIDEQLLGFRGRCPFRVYIPNKPSKYG |
| | IKILMMCDSGTKYMINGMPYLGRGTQTNGVPLGEYYVKELSKPVHGSC |
| | RNITCDNWFTSIPLAKNLLQEPYKLTIVGTVASNAREIPEVLKNSRSRPVG |
| | TSMFCFDGPLTLVSYKPKPAKMVYLLSSCDEDASINESTGKPQMVMYY |
| | NQTKGGVDTLNQMCSVMTCSRKTNRWPMALLYGMINIACINSFIIYSHN |
| | VSSKGEKVQSRKKFMRNLYMSLTSSFMRKRLEAPTLKRYLRDNISNILP |
| | KEVPGTSDDSTEEPVMKKRTYCTYCPSKIRRKANASCKKCKKVICREHN |
| | IDMCQSCF (SEQ ID NO: 15) |
| PBx | KNKHCWSTSKSTRRSRVSALNIVRSQRGPTRMCRNIYDPLLCFKLFFTDE |
| Delta 92 | IISEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| | KILMMCDSGTKYMINGMPYLGRGTQTNGVPLGEYYVKELSKPVHGSCR |
| | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCF (SEQ ID NO: 16) |
| PBx | NKHCWSTSKSTRRSRVSALNIVRSQRGPTRMCRNIYDPLLCFKLFFTDEII |
| Delta 93 | SEIVKWTNAEISLKRRESMTSATFRDTNEDEIYAFFGILVMTAVRKDNH |
| N- | MSTDDLFDRSLSMVYVSVMSRDRFDFLIRCLRMDDKSIRPTLRENDVFT |
| Terminal | PVRKIWDLFIHQCIQNYTPGAHLTIDEQLLGFRGRCPFRVYIPNKPSKYGI |
| | KILMMCDSGTKYMINGMPYLGRGTQTNGVPLGEYYVKELSKPVHGSCR |
| | NITCDNWFTSIPLAKNLLQEPYKLTIVGTVASNAREIPEVLKNSRSRPVGT |
| | SMFCFDGPLTLVSYKPKPAKMVYLLSSCDEDASINESTGKPQMVMYYN |
| | QTKGGVDTLNQMCSVMTCSRKTNRWPMALLYGMINIACINSFIIYSHNV |
| | SSKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCF (SEQ ID NO: 17) |
| PBx | KHCWSTSKSTRRSRVSALNIVRSQRGPTRMCRNIYDPLLCFKLFFTDEIIS |
| Delta 94 | EIVKWTNAEISLKRRESMTSATFRDTNEDEIYAFFGILVMTAVRKDNHM |
| N- | STDDLFDRSLSMVYVSVMSRDRFDFLIRCLRMDDKSIRPTLRENDVFTPV |
| Terminal | RKIWDLFIHQCIQNYTPGAHLTIDEQLLGFRGRCPFRVYIPNKPSKYGIKI |
| | LMMCDSGTKYMINGMPYLGRGTQTNGVPLGEYYVKELSKPVHGSCRNI |
| | TCDNWFTSIPLAKNLLQEPYKLTIVGTVASNAREIPEVLKNSRSRPVGTS |
| | MFCFDGPLTLVSYKPKPAKMVYLLSSCDEDASINESTGKPQMVMYYNQ |
| | TKGGVDTLNQMCSVMTCSRKTNRWPMALLYGMINIACINSFIIYSHNVS |
| | SKGEKVQSRKKFMRNLYMSLTSSFMRKRLEAPTLKRYLRDNISNILPKE |
| | VPGTSDDSTEEPVMKKRTYCTYCPSKIRRKANASCKKCKKVICREHNID |
| | MCQSCF (SEQ ID NO: 18) |
| PBx | HCWSTSKSTRRSRVSALNIVRSQRGPTRMCRNIYDPLLCFKLFFTDEIISEI |
| Delta 95 | VKWTNAEISLKRRESMTSATFRDTNEDEIYAFFGILVMTAVRKDNHMST |
| N- | DDLFDRSLSMVYVSVMSRDRFDFLIRCLRMDDKSIRPTLRENDVFTPVR |
| Terminal | KIWDLFIHQCIQNYTPGAHLTIDEQLLGFRGRCPFRVYIPNKPSKYGIKIL |
| | MMCDSGTKYMINGMPYLGRGTQTNGVPLGEYYVKELSKPVHGSCRNIT |
| | CDNWFTSIPLAKNLLQEPYKLTIVGTVASNAREIPEVLKNSRSRPVGTSM |
| | FCFDGPLTLVSYKPKPAKMVYLLSSCDEDASINESTGKPQMVMYYNQT |
| | KGGVDTLNQMCSVMTCSRKTNRWPMALLYGMINIACINSFIIYSHNVSS |
| | KGEKVQSRKKFMRNLYMSLTSSFMRKRLEAPTLKRYLRDNISNILPKEV |
| | PGTSDDSTEEPVMKKRTYCTYCPSKIRRKANASCKKCKKVICREHNIDM |
| | CQSCF (SEQ ID NO: 19) |
| PBx | CWSTSKSTRRSRVSALNIVRSQRGPTRMCRNIYDPLLCFKLFFTDEIISEIV |
| Delta 96 | KWTNAEISLKRRESMTSATFRDTNEDEIYAFFGILVMTAVRKDNHMSTD |
| N- | DLFDRSLSMVYVSVMSRDRFDFLIRCLRMDDKSIRPTLRENDVFTPVRKI |
| Terminal | WDLFIHQCIQNYTPGAHLTIDEQLLGFRGRCPFRVYIPNKPSKYGIKILM |
| | MCDSGTKYMINGMPYLGRGTQTNGVPLGEYYVKELSKPVHGSCRNITC |
| | DNWFTSIPLAKNLLQEPYKLTIVGTVASNAREIPEVLKNSRSRPVGTSMF |
| | CFDGPLTLVSYKPKPAKMVYLLSSCDEDASINESTGKPQMVMYYNQTK |
| | GGVDTLNQMCSVMTCSRKTNRWPMALLYGMINIACINSFIIYSHNVSSK |
| | GEKVQSRKKFMRNLYMSLTSSFMRKRLEAPTLKRYLRDNISNILPKEVP |
| | GTSDDSTEEPVMKKRTYCTYCPSKIRRKANASCKKCKKVICREHNIDMC |
| | QSCF (SEQ ID NO: 20) |
| PBx | WSTSKSTRRSRVSALNIVRSQRGPTRMCRNIYDPLLCFKLFFTDEIISEIVK |
| Delta 97 | WTNAEISLKRRESMTSATFRDTNEDEIYAFFGILVMTAVRKDNHMSTDD |
| N- | LFDRSLSMVYVSVMSRDRFDFLIRCLRMDDKSIRPTLRENDVFTPVRKIW |
| Terminal | DLFIHQCIQNYTPGAHLTIDEQLLGFRGRCPFRVYIPNKPSKYGIKILMMC |
| | DSGTKYMINGMPYLGRGTQTNGVPLGEYYVKELSKPVHGSCRNITCDN |
| | WFTSIPLAKNLLQEPYKLTIVGTVASNAREIPEVLKNSRSRPVGTSMFCF |
| | DGPLTLVSYKPKPAKMVYLLSSCDEDASINESTGKPQMVMYYNQTKGG |
| | VDTLNQMCSVMTCSRKTNRWPMALLYGMINIACINSFIIYSHNVSSKGE |
| | KVQSRKKFMRNLYMSLTSSFMRKRLEAPTLKRYLRDNISNILPKEVPGTS |
| | DDSTEEPVMKKRTYCTYCPSKIRRKANASCKKCKKVICREHNIDMCQSC |
| | F (SEQ ID NO: 21) |
| PBx | STSKSTRRSRVSALNIVRSQRGPTRMCRNIYDPLLCFKLFFTDEIISEIVKW |
| Delta 98 | TNAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| | SGTKYMINGMPYLGRGTQTNGVPLGEYYVKELSKPVHGSCRNITCDNW |
| | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCF |
| | (SEQ ID NO: 22) |
| PBx | TSKSTRRSRVSALNIVRSQRGPTRMCRNIYDPLLCFKLFFTDEIISEIVKWT |
| Delta 99 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| | SGTKYMINGMPYLGRGTQTNGVPLGEYYVKELSKPVHGSCRNITCDNW |
| | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCF |
| | (SEQ ID NO: 23) |
| PBx | SKSTRRSRVSALNIVRSQRGPTRMCRNIYDPLLCFKLFFTDEIISEIVKWT |
| Delta 100 | NAEISLKRRESMTSATFRDTNEDEIYAFFGILVMTAVRKDNHMSTDDLF |
| N- | DRSLSMVYVSVMSRDRFDFLIRCLRMDDKSIRPTLRENDVFTPVRKIWD |
| Terminal | LFIHQCIQNYTPGAHLTIDEQLLGFRGRCPFRVYIPNKPSKYGIKILMMCD |
| | SGTKYMINGMPYLGRGTQTNGVPLGEYYVKELSKPVHGSCRNITCDNW |
| | FTSIPLAKNLLQEPYKLTIVGTVASNAREIPEVLKNSRSRPVGTSMFCFDG |
| | PLTLVSYKPKPAKMVYLLSSCDEDASINESTGKPQMVMYYNQTKGGVD |
| | TLNQMCSVMTCSRKTNRWPMALLYGMINIACINSFIIYSHNVSSKGEKV |
| | QSRKKFMRNLYMSLTSSFMRKRLEAPTLKRYLRDNISNILPKEVPGTSDD |
| | STEEPVMKKRTYCTYCPSKIRRKANASCKKCKKVICREHNIDMCQSCF |
| | (SEQ ID NO: 24) |
| PBx | KSTRRSRVSALNIVRSQRGPTRMCRNIYDPLLCFKLFFTDEIISEIVKWTN |
| Delta 101 | AEISLKRRESMTSATFRDTNEDEIYAFFGILVMTAVRKDNHMSTDDLFD |
| N- | RSLSMVYVSVMSRDRFDFLIRCLRMDDKSIRPTLRENDVFTPVRKIWDLF |
| Terminal | IHQCIQNYTPGAHLTIDEQLLGFRGRCPFRVYIPNKPSKYGIKILMMCDSG |
| | TKYMINGMPYLGRGTQTNGVPLGEYYVKELSKPVHGSCRNITCDNWFT |
| | SIPLAKNLLQEPYKLTIVGTVASNAREIPEVLKNSRSRPVGTSMFCFDGPL |
| | TLVSYKPKPAKMVYLLSSCDEDASINESTGKPQMVMYYNQTKGGVDTL |
| | NQMCSVMTCSRKTNRWPMALLYGMINIACINSFIIYSHNVSSKGEKVQS |
| | RKKFMRNLYMSLTSSFMRKRLEAPTLKRYLRDNISNILPKEVPGTSDDST |
| | EEPVMKKRTYCTYCPSKIRRKANASCKKCKKVICREHNIDMCQSCF |
| | (SEQ ID NO: 25) |
| PBx | STRRSRVSALNIVRSQRGPTRMCRNIYDPLLCFKLFFTDEIISEIVKWTNA |
| Delta 102 | EISLKRRESMTSATFRDTNEDEIYAFFGILVMTAVRKDNHMSTDDLFDRS |
| N- | LSMVYVSVMSRDRFDFLIRCLRMDDKSIRPTLRENDVFTPVRKIWDLFIH |
| Terminal | QCIQNYTPGAHLTIDEQLLGFRGRCPFRVYIPNKPSKYGIKILMMCDSGT |
| | KYMINGMPYLGRGTQTNGVPLGEYYVKELSKPVHGSCRNITCDNWFTSI |
| | PLAKNLLQEPYKLTIVGTVASNAREIPEVLKNSRSRPVGTSMFCFDGPLT |
| | LVSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCF (SEQ |
| | ID NO: 26) |
| PBx | TRRSRVSALNIVRSQRGPTRMCRNIYDPLLCFKLFFTDEIISEIVKWTNAEI |
| Delta 103 | SLKRRESMTSATFRDTNEDEIYAFFGILVMTAVRKDNHMSTDDLFDRSL |
| N- | SMVYVSVMSRDRFDFLIRCLRMDDKSIRPTLRENDVFTPVRKIWDLFIHQ |
| Terminal | CIQNYTPGAHLTIDEQLLGFRGRCPFRVYIPNKPSKYGIKILMMCDSGTK |
| | YMINGMPYLGRGTQTNGVPLGEYYVKELSKPVHGSCRNITCDNWFTSIP |
| | LAKNLLQEPYKLTIVGTVASNAREIPEVLKNSRSRPVGTSMFCFDGPLTL |
| | VSYKPKPAKMVYLLSSCDEDASINESTGKPQMVMYYNQTKGGVDTLN |
| | QMCSVMTCSRKTNRWPMALLYGMINIACINSFIIYSHNVSSKGEKVQSR |
| | KKFMRNLYMSLTSSFMRKRLEAPTLKRYLRDNISNILPKEVPGTSDDSTE |
| | EPVMKKRTYCTYCPSKIRRKANASCKKCKKVICREHNIDMCQSCF (SEQ |
| | ID NO: 27) |

### DNA Targeting Domains

The transposase domains and fusion proteins provided herein may further comprise one or more DNA targeting domains. A DNA-targeting domain may be attached to the C-terminus or the N-terminus of the transposase domain or the fusion protein. In some embodiments, the DNA-targeting domain is attached to the N-terminus of the transposase domain, e.g., a transposase domain comprising an N-terminal deletion. Without wishing to be bound by theory, it is believed that addition a DNA targeting domain to a transposase domain improves site-specific transposase activity by targeting the transposase fused to the DNA targeting domain to the targeted site. In some embodiments, the insertion of a DNA targeting domain improves site-specific transposase activity by at least 2-fold, at least 3-fold, at least 4-fold, or at least 5-fold compared to the same transposase domain not comprising a DNA targeting domain.

Any DNA targeting domain known in the art may be used in the context of the transposase domains, fusion proteins, and tandem dimer transposases described herein, including, without limitation, CRISPR, Zinc Finger Motifs, TALE, and transcription factors. In some embodiments, the DNA targeting domain comprises one, two or three Zinc Finger Motifs. In some embodiments, the DNA targeting domain comprises three Zinc Finger Motifs. In some embodiments, the three Zinc Finger Motifs are flanked by GGGGS SEQ ID NO: 181) linkers. In some embodiments, the three Zinc Finger Motifs flanked by GGGGS (SEQ ID NO: 181) linkers cumulatively comprise the sequence set forth in SEQ ID NO: 28: GGGGSERPYACPVESCDRRFSRSDELTRHIRIHTGQKPFQCRICMRNFSRSDHLTTHIRTH TGEKPFACDICGRKFARSDERKRHTKIHLRQKDGGGGS (SEQ ID NO: 28) or a sequence having at least 75%, at least 80%, at least 85%, at least 90%, at least 95%, at least 98%, or at least 99% identity thereto.

In a specific embodiment, provided herein is a fusion protein comprising a transposase domain comprising an N-terminal deletion, an NLS, and three Zinc Finger Motifs. In some embodiments, the NLS comprises or consists of the sequence set forth in SEQ ID NO: 29.

In some aspects, the DNA targeting domain is a TAL array. TALEs (Transcription activator-like effectors) from Xanthomonas typically contain a 288 amino acid N-terminus followed by an array of a variable number of ˜34 amino acid repeats followed by a 278 amino acid C-terminus (SEQ ID NO: 30); however, truncated versions have been described in the literature (e.g., see Miller et al., Nat Biotechnol 29, 143-148 (2011). TALs fused to a FokI nuclease (called TALENs) most often contain truncations of the N and C terminus. For example, the first 152 amino acids of the N-terminus is often removed (called Delta 152; SEQ ID No 31) and the C-terminus is often truncated leaving 63 amino acids (called +63; SEQ ID NO: 32).

TALs contain arrays of 34 amino acids repeated a variable number of times. The two amino acids at position 12 and 13 are varied and determine which nucleotide the TAL repeat will recognize. This feature allows a TAL array to be programed to bind a specific DNA sequence. The amino acids NG recognize T, NI recognize A, NN recognize G or A, HD recognize C, NK recognize G, NS recognize A, C, G or T. Other amino acids within the 34 residue repeat may also be varied. For example, position 11 is often changed to an N for repeats that recognize G. Also, positions 4 and 32 are often varied to reduce the repetitiveness of the array but not to determine the binding specificity. The number of 34 amino acid repeats in an array determines the length of the DNA sequence recognized (one protein repeat binds one DNA bp). Furthermore, the last bp is recognized by a “half array” that is 20 amino acids rather than 34.

In addition, the N-terminal domain of TALs (e.g., SEQ ID NO: 31) recognizes and requires a T that is located immediately 5′ of the target DNA sequence. Mutations of TAL N-terminal domains have been described in the literature that no longer require a 5′ T (Lamb et al., Nucleic Acids Res. 2013 November; 41(21):9779-85). For example, the NT-G mutant requires a 5′G instead of a 5′T (SEQ ID NO: 33) while the NT-βN mutant does not require any specific 5′ nucleotide (SEQ ID NO: 34). These mutated N-terminal domain sequences may be used to provide additional sequence options that may be targeted using TAL Arrays.

In general, each TAL array comprises nine 34-amino acid repeats followed by the 20 amino acid “half” repeat. TAL arrays may be synthesized with flanking BsmBI type IIS restriction sites. In one embodiment, individual TAL modules containing 34 amino acid or 20 amino acid “half” repeats may be designed and synthesized flanked by BsmBI type IIS restriction sites. The entire TAL module set contains 4 modules capable of recognizing either A, C, G, T for each of 10 bp positions (40 modules/10 bp target), and one TAL half repeat module. Illustrative TAL modules are set forth in SEQ ID NOs: 35-38, wherein X is any amino acid:

•TAL Module Version 1:
(SEQ ID NO: 35)
LTPDQVVAIAXXXGGKQALETVQRLLPVLCQDHG
•TAL Module Version 2:
(SEQ ID NO: 36)
LTPEQVVAIAXXXGGKQALETVQRLLPVLCQAHG
•TAL Module Version 3”
(SEQ ID NO: 37)
LTPDQVVAIAXXXGGKQALETVQRLLPVLCQAHG
•TAL Module Version 4:
(SEQ ID NO: 38)
LTPAQVVAIAXXXGGKQALETVQRLLPVLCQDHG.

An exemplary TAL Half Module is set forth in SEQ ID NO: 39, wherein X is any amino acid: LTPEQVVAIAXXXGGRPALE (SEQ ID NO: 39).

Pairs of TAL arrays targeting sequences in the desired gene may be designed and the corresponding modules selected and pooled together using “Golden Gate Assembly,” to assemble in frame each TAL-Array. The DNA sequence encoding TAL Arrays generated herein may be further codon optimized using GeneArt algorithms (Thermo Fisher™)

When designing left and right TAL Arrays comprising a N-terminal domain recognizing a T and a TAL C-terminal domain to be fused to an N-terminal deleted transposase sequence (i.e., TAL-ssSPB or TAL-PBx; described below), one TAL Array recognizes a sequence 5′ of the TTAA and the other TAL Array recognizes a sequence 3′ of the TTAA. Since the sequence 5′ of TTAA is most often different from the sequence 3′ of TTAA in genomic DNA targets, TAL-ssSPB will most often be used as a heterodimer consisting of two different TAL domains that recognize two different DNA sequences. Additionally, the sequence recognized by the TAL Array is not directly adjacent to the TTAA. Instead, it is separated from the TTAA by a spacer of a given bp length, e.g., spacers of 12 bp, 13 bp or 14 bp.

A TAL array may target any DNA sequence (e.g., genomic DNA sequence) of interest. It will be apparent to a person of skill in the art that any left TAL array for a given target can be combined with any right TAL array for the same target.

In some embodiments, a TAL array targets green fluorescent protein (GFP). TAL-piggyBac transposase fusion proteins comprising N-terminal deleted piggyBac transposase sequences and integration defective N-terminal piggyBac transposase targeting GFP have been described in co-owned International Patent Application Publication No. PCT/2022/22549.

In some embodiments, a TAL array targets an LPA gene repeat element. Illustrative sequences of left TAL arrays targeting an LPA repeat element are set forth in SEQ ID NOs: 116, 118, 121, 124, 125, 127, 129, 131, 133, 135, 137, 139 and 141. Illustrative sequences of right TAL arrays targeting LPA are set forth in SEQ ID NOs: 117, 119, 120, 122, 123, 126, 128, 130, 132, 134, 136, 138, 140, and 142. In some embodiments, the left TAL array targeting an LPA repeat element binds to a nucleic acid molecule comprising the sequence set forth in SEQ ID NOs: 89, 91, 94, 97, 98, 100, 102, 104, 106, 108, 110, 112, and 114. In some embodiments, the right TAL array targeting an LPA repeat element binds to a nucleic acid molecule comprising the sequence set forth in SEQ ID NOs: 90, 92, 93, 95, 96, 99, 101, 103, 105, 107, 109, 111, 113, and 115. It will be apparent to a person of skill in the art that any left TAL array disclosed herein may be combined with any right TAL array disclosed herein. Illustrative genomic target sites for an LPA repeat elements are set forth in SEQ ID NOs: 81-88.

The present disclosure provides fusion proteins comprising a DNA targeting domain attached to the transposase domain in different ways. In some embodiments, the DNA targeting domain may be fused or linked to the N-terminus of a transposase domain comprising an N-terminal deletion. For example, the DNA targeting domain may be inserted into a transposase domain at a suitable position in the N-terminal region of the transposase domain. In some embodiments, the DNA targeting domain may replace one or more amino acid(s) in the N-terminal region of the transposase domain. In some embodiments, the DNA targeting domain is inserted into a transposase domain at a suitable position in the N-terminal region of the transposase domain without replacing an amino acid.

The DNA targeting domain may be inserted into the N-terminus of a transposase domain. For example, the DNA targeting domain is inserted into the N-terminus of the transposase domain at a position after the 82^{nd} amino acid and before the 105^{th} amino acid of SEQ ID NO: 4. In some embodiments, the DNA targeting domain is inserted between the 82^{nd} and 83^{rd} amino acid of SEQ ID NO: 2 or 3 (with numbering beginning from the 5th or 12th amino acid respectively) or of SEQ ID NO: 4. In some embodiments, the DNA targeting domain is inserted between the 83^{rd} and 84^{th} amino acid of SEQ ID NO: 2 or 3 (with numbering beginning from the 5th or 12th amino acid respectively) or of SEQ ID NO: 4. In some embodiments, the DNA targeting domain is inserted between the 84^{th} and 85^{th} amino acid of SEQ ID NO: 2 or 3 (with numbering beginning from the 5th or 12th amino acid respectively) or of SEQ ID NO: 4. In some embodiments, the DNA targeting domain is inserted between the 85^{th} and 86^{th} amino acid of SEQ ID NO: 2 or 3 (with numbering beginning from the 5th or 12th amino acid respectively) or of SEQ ID NO: 4. In some embodiments, the DNA targeting domain is inserted between the 86^{th} and 87^{th} amino acid of SEQ ID NO: 2 or 3 (with numbering beginning from the 5th or 12th amino acid respectively) or of SEQ ID NO: 4. In some embodiments, the DNA targeting domain is inserted between the 87^{th} and 88^{th} amino acid of SEQ ID NO: 2 or 3 (with numbering beginning from the 5th or 12th amino acid respectively) or of SEQ ID NO: 4. In some embodiments, the DNA targeting domain is inserted between the 88^{th} and 89^{th} amino acid of SEQ ID NO: 2 or 3 (with numbering beginning from the 5th or 12th amino acid respectively) or of SEQ ID NO: 4. In some embodiments, the DNA targeting domain is inserted between the 89^{th} and 90^{th} amino acid of SEQ ID NO: 2 or 3 (with numbering beginning from the 5th or 12th amino acid respectively) or of SEQ ID NO: 4. In some embodiments, the DNA targeting domain is inserted between the 90^{th} and 91^{st} amino acid of SEQ ID NO: 2 or 3 (with numbering beginning from the 5th or 12th amino acid respectively) or of SEQ ID NO: 4. In some embodiments, the DNA targeting domain is inserted between the 91^{st} and 92^{nd} amino acid of SEQ ID NO: 2 or 3 (with numbering beginning from the 5th or 12th amino acid respectively) or of SEQ ID NO: 4. In some embodiments, the DNA targeting domain is inserted between the 92^{nd} and 93^{rd} amino acid of SEQ ID NO: 2 or 3 (with numbering beginning from the 5th or 12th amino acid respectively) or of SEQ ID NO: 4. In some embodiments, the DNA targeting domain is inserted between the 93^{rd} and 94^{th} amino acid of SEQ ID NO: 2 or 3 (with numbering beginning from the 5th or 12th amino acid respectively) or of SEQ ID NO: 4. In some embodiments, the DNA targeting domain is inserted between the 94^{th} and 95^{th} amino acid of SEQ ID NO: 2 or 3 (with numbering beginning from the 5th or 12th amino acid respectively) or of SEQ ID NO: 4. In some embodiments, the DNA targeting domain is inserted between the 95^{th} and 96^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 96^{th} and 97^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 97^{th} and 98^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 98^{th} and 99^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 99^{th} and 100^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 100^{th} and 101^{st} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 101^{st} and 102^{nd} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 102^{nd} and 103^{rd} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 103^{rd} and 104^{th} amino acid of SEQ ID NO: 2 or 3 (with numbering beginning from the 5^{th} or 12^{th} amino acid respectively) or of SEQ ID NO: 4. In some embodiments, the DNA targeting domain is inserted between the 104 and 105^{th} amino acid of SEQ ID NO: 2 or 3 (with numbering beginning from the 5^{th} or 12^{th} amino acid respectively) or of SEQ ID NO: 4. In some embodiments, the DNA targeting domain comprises the sequence of SEQ ID NO: 28 or a sequence having at least 75%, at least 80%, at least 85%, at least 90%, at least 95%, at least 98%, or at least 99% identity thereto. The transposase domain may further comprise an NLS, for example, and NLS of SEQ ID NO: 29.

The DNA targeting domain may replace one or more amino acid(s) in the N-terminal region of the transposase domain. For example, the DNA targeting domain may replace one or more amino acid(s) in the transposase domain between, and including, the 83^{rd} amino acid and the 105^{th} amino acid of SEQ ID NO: 2 or 3 (with numbering beginning from the 5^{th} or 12^{th} amino acid, respectively) or of SEQ ID NO: 4. In some embodiments, the DNA targeting domain replaces the 83^{rd} amino acid of SEQ ID NO: 2 or 3 (with numbering beginning from the 5^{th} or 12^{th} amino acid respectively) or of SEQ ID NO: 4. In some embodiments, the DNA targeting domain replaces the 84^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 85^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 86^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 87^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 88^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 89^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 90^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 91^{st} amino acid of SEQ ID NO: 2 or 3 (with numbering beginning from the 5^{th} or 12^{th} amino acid respectively) or of SEQ ID NO: 4. In some embodiments, the DNA targeting domain replaces the 92^{nd} amino acid of SEQ ID NO: 2 or 3 (with numbering beginning from the 5^{th} or 12^{th} amino acid respectively) or of SEQ ID NO: 4. In some embodiments, the DNA targeting domain replaces the 93^{rd} amino acid of SEQ ID NO: 2 or 3 (with numbering beginning from the 5^{th} or 12^{th} amino acid respectively) or of SEQ ID NO: 4. In some embodiments, the DNA targeting domain replaces the 94^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 95^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 96^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 97^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 98^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 99^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 100^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 101^{st} amino acid of SEQ ID NO: 2 or 3 (with numbering beginning from the 5^{th} or 12^{th} amino acid respectively) or of SEQ ID NO: 4. In some embodiments, the DNA targeting domain replaces the 102^{nd} amino acid of SEQ ID NO: 2 or 3 (with numbering beginning from the 5^{th} or 12^{th} amino acid respectively) or of SEQ ID NO: 4. In some embodiments, the DNA targeting domain replaces the 103^{rd} amino acid of SEQ ID NO: 2 or 3 (with numbering beginning from the 5^{th} or 12^{th} amino acid respectively) or of SEQ ID NO: 4. In some embodiments, the DNA targeting domain replaces the 104^{th} amino acid of SEQ ID NO: 2 or 3 (with numbering beginning from the 5^{th} or 12^{th} amino acid respectively) or of SEQ ID NO: 4. In some embodiments, the DNA targeting domain replaces the 105^{th} amino acid of SEQ ID NO: 2 or 3 (with numbering beginning from the 5^{th} or 12^{th} amino acid respectively) or of SEQ ID NO: 4. In some embodiments, the DNA targeting domain comprises the sequence of SEQ ID NO: 28 or a sequence having at least 75%, at least 80%, at least 85%, at least 90%, at least 95%, at least 98%, or at least 99% identity thereto. The transposase domain may further comprise an NLS, for example, an NLS of SEQ ID NO: 29.

An illustrative sequence of a fusion protein comprising a transposase domain comprising an N-terminal deletion of 93 amino acids, an NLS, and three Zinc Finger Motifs flanked by GGGGS (SEQ ID NO: 181) linkers is show in SEQ ID NO: 40, where the NLS is shown in italics, the sequence comprising the three Zinc Finger Motifs and GGGGS linkers is underlined, and the transposase domain comprising an N-terminal deletion of 93 amino acid is shown in bold:

(SEQ ID NO: 40)
MAPKKKRKVGGGGSERPYACPVESCDRRFSRSDELTRHIRIHTGQKPFQCRICM
RNFSRSDHLTTHIRTHTGEKPFACDICGRKFARSDERKRHTKIHLRQKDGGGGSNKHCW
STSKSTRRSRVSALNIVRSQRGPTRMCRNIYDPLLCFKLFFTDEIISEIVKWTNAEISL
KRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQ
LLGFRGRCPFRVYIPNKPSKYGIKILMMCDSGTKYMINGMPYLGRGTQTNGVPLG
EYYVKELSKPVHGSCRNITCDNWFTSIPLAKNLLQEPYKLTIVGTVASNAREIPEVL
KNSRSRPVGTSMFCFDGPLTLVSYKPKPAKMVYLLSSCDEDASINESTGKPQMVMY
YNQTKGGVDTLNQMCSVMTCSRKTNRWPMALLYGMINIACINSFIIYSHNVSSKGE
KVQSRKKFMRNLYMSLTSSFMRKRLEAPTLKRYLRDNISNILPKEVPGTSDDSTEE
PVMKKRTYCTYCPSKIRRKANASCKKCKKVICREHNIDMCQSCF

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 40. In some embodiments, a fusion protein described herein comprises a transposase domain comprising the amino acid sequence set forth in SEQ ID NO: 40 with one, two, three, four or five conservative amino acid substitutions. In some embodiments, a fusion protein described herein comprises a transposase domain comprising the amino acid sequence set forth in SEQ ID NO: 40. An illustrative sequence of a fusion protein comprising an integration deficient transposase domain comprising an N-terminal deletion of 93 amino acids, an NLS, and three Zinc Finger Motifs flanked by GGGGS (SEQ ID NO: 181) linkers is set forth in SEQ ID NO: 180, where the NLS is shown in italics, the sequence comprising the three Zinc Finger Motifs and GGGGS linkers is underlined, and the transposase domain comprising an N-terminal deletion of 93 amino acid is shown in bold:

(SEQ ID NO: 180)
MAPKKKRKVGGGGSERPYACPVESCDRRFSRSDELTRHIRIHTGQKPFQCRICM
RNFSRSDHLTTHIRTHTGEKPFACDICGRKFARSDERKRHTKIHLRQKDGGGGSNKHCW
STSKSTRRSRVSALNIVRSQRGPTRMCRNIYDPLLCFKLFFTDEIISEIVKWTNAEISL
KRRESMTSATFRDTNEDEIYAFFGILVMTAVRKDNHMSTDDLFDRSLSMVYVSVMS
RDRFDFLIRCLRMDDKSIRPTLRENDVFTPVRKIWDLFIHQCIQNYTPGAHLTIDEQ
LLGFRGRCPFRVYIPNKPSKYGIKILMMCDSGTKYMINGMPYLGRGTQTNGVPLG
EYYVKELSKPVHGSCRNITCDNWFTSIPLAKNLLQEPYKLTIVGTVASNAREIPEVL
KNSRSRPVGTSMFCFDGPLTLVSYKPKPAKMVYLLSSCDEDASINESTGKPQMVMY
YNQTKGGVDTLNQMCSVMTCSRKTNRWPMALLYGMINIACINSFIIYSHNVSSKGE
KVQSRKKFMRNLYMSLTSSFMRKRLEAPTLKRYLRDNISNILPKEVPGTSDDSTEE
PVMKKRTYCTYCPSKIRRKANASCKKCKKVICREHNIDMCQSCF.

In some embodiments, a fusion protein described herein comprises a transposase domain comprising an amino acid sequence that is at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 180. In some embodiments, a fusion protein described herein comprises a transposase domain comprising the amino acid sequence set forth in SEQ ID NO: 180 with one, two, three, four or five conservative amino acid substitutions. In some embodiments, a fusion protein described herein comprises a transposase domain comprising the amino acid sequence set forth in SEQ ID NO: 180.

### Nuclear Localization Signals

In some embodiments, the transposase domains and fusion proteins provided herein may comprise an in-frame nuclear localization sequence (NLS). Examples of transposases fused to a nuclear localization signal are disclosed in U.S. Pat. Nos. 6,218,185; 6,962,810, 8,399,643 and WO 2019/173636, each of which is incorporated herein by reference in its entirety for examples of transposase domains that may be used in the fusion proteins described herein. In some embodiments, the NLS comprises the sequence of PKKKRKV (SEQ ID NO: 29). In certain aspects, the in-frame NLS is located upstream (N-terminal) of the transposase domain comprising an N-terminal deletion.

In general, the NLS is preferably located at the N-terminal end of a fusion protein. In some embodiments, the NLS is fused or linked to the N-terminus of a transposase domain. In some embodiments, the NLS is fused or linked to the N-terminus of a DNA targeting domain.

In certain aspects, the in-frame NLS is fused directly to the amino terminus of the transposase domain comprising an N-terminal deletion. In some embodiments, the NLS is attached to the N-terminus of a transposase domain comprising an N-terminal deletion via a linker (e.g., a GGGGS linker or a GGS linker).

In some embodiments, an initiator methionine is introduced before the NLS. In some embodiments, additional alanine residues are introduced before and/or after the NLS to ensure in-frame translation. As such, the numbering of the residues in SEQ ID NOs: 1 and 3 begins at the 12^{th} residue of SEQ ID NOs: 1 and 3 for the purpose of identifying deleted and mutated residues. In SEQ ID NO: 2, which is the sequence of SPB, which does not comprise an NLS, the numbering of residues begins at the 5^{th} residue for the purpose of identifying deleted and mutated residues. In SEQ ID NO: 4, the numbering begins at the first residue for the purpose of identifying deleted and mutated residues.

In some embodiments, a fusion protein comprises an NLS and a transposase domain comprising an N-terminal deletion of 93 amino acids. In some embodiments, the fusion protein comprises an amino acid sequence that is at least 75%, at least 80%, at least 85%, at least 90%, at least 95%, at least 98% or at least 99% identical to the amino acid sequence set forth in SEQ ID NO: 5. In some embodiments, the fusion protein comprises the amino acid sequence set forth in SEQ ID NO: 5.

### Obligate Heterodimers and Tandem Dimers

In another aspect, provided herein are tandem dimer transposases comprising two fusion proteins, each fusion protein comprising a transposase domain and one or both fusion proteins further comprising a DNA targeting domain. In some embodiments, both fusion proteins comprise a DNA targeting domain. In some embodiments, both fusion proteins comprise DNA targeting domains and the DNA targeting domains target DNA sequences that are adjacent to the DNA sequence which is the insertion site targeted by the transposase. In some embodiments, only one of the two fusion proteins in the tandem dimer transposase comprises a DNA targeting domain. A DNA-targeting domain may be attached to the C-terminus or the N-terminus of the fusion protein.

Thus, in some embodiments, provided herein is a complex comprising (a) a first fusion protein comprising a first transposase domain and a first DNA targeting domain; and (b) a second fusion protein comprising a first transposase domain and a second DNA targeting domain, wherein the first DNA targeting domain and the second DNA targeting domain are different; wherein the transposase domain of the first fusion protein and the transposase domain of the second fusion protein have opposing charge that permits the two fusion proteins to form a complex.

In some embodiments, provided herein is a complex comprising (a) a first fusion protein comprising, in N-terminal to C-terminal order: a first NLS, a first DNA targeting domain, and a first transposase domain comprising an N-terminal deletion; and (b) a second fusion protein comprising in N-terminal to C-terminal order: a second NLS, a second DNA targeting domain, and a second transposase domain comprising an N-terminal deletion; wherein the transposase domain of the first fusion protein and the transposase domain of the second fusion protein have opposing charge that permits the two fusion proteins to form a complex. In some embodiments, the first and/or second transposase domains are SPB domains. In some embodiments, the first and/or second transposase domains are PBx transposase domains. In some embodiments, the first and/or second transposase domain comprises an N-terminal deletion of 83, 84, 85, 86, 87, 88, 89, 90, 91, 92, 93, 94, 95, 96, 97, 98, 99, 100, 101, 102, or 103 amino acids. In some embodiments, the first and second transposase domains comprise the sequence of SEQ ID NO: 5 or 6. In some embodiments, the first and/or second DNA targeting domain comprises one, two or three Zinc Fingers Motifs. In some embodiments, the first and/or second DNA targeting domain comprises the sequence of SEQ ID NO: 28. In some embodiment, the first and/or second DNA targeting domain comprises TAL motifs.

In some embodiments, provided herein is a complex comprising (a) a first fusion protein comprising, in N-terminal to C-terminal order: a first NLS and a first transposase domain comprising the sequence of SEQ ID NO: 2, 3, or 4; and (b) a second fusion protein comprising in N-terminal to C-terminal order: a second NLS and a second transposase domain comprising the sequence of SEQ ID NO: 2, 3, or 4; wherein the first and the second transposase domain comprise a DNA targeting domain, and wherein the transposase domain of the first fusion protein and the transposase domain of the second fusion protein have opposing charge that permits the two fusion proteins to form a complex. In some embodiment, the first and/or second DNA targeting domain comprises one, two or three Zinc Fingers Motifs. In some embodiments, the first and/or second DNA targeting domain comprises the sequence of SEQ ID NO: 28. In some embodiments, the first and/or second DNA targeting domain comprises TAL motifs. In some embodiments, the first DNA targeting domain replaces one or more amino acid(s) between, and including, the 83^{rd} amino acid and the 105^{th} amino acid of the first transposase domain, with numbering beginning at residue 5 or 12 of SEQ ID NO: 2 or 3 respectively. In some embodiments, the first DNA targeting domain replaces the 83^{rd} 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd}, or 103^{rd} residue of the first transposase domain, with numbering beginning at residue 5 or 12 of SEQ ID NO: 2 or 3 respectively. In some embodiments, the first DNA targeting domain replaces one or more amino acid(s) between, and including, the 83^{rd} amino acid and the 105^{th} amino acid of the second transposase domain, with numbering beginning at residue 5 or 12 of SEQ ID NO: 2 or 3 respectively. In some embodiments, the second DNA targeting domain replaces the 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th}, 99^{th}, 100^{th}, 101^{st}, 102^{nd} or 103^{rd} residue of the second transposase domain, with numbering beginning at residue 5 or 12 of SEQ ID NO: 2 or 3 respectively.

In another aspect, provided herein are fusion proteins comprising a transposase domain that can form obligate heterodimers with another fusion protein comprising a transposase domain. Without wishing to be bound by theory, it is believed that two such fusion proteins assemble into a dimer structure held together through a combination of charge interactions, hydrogen bonds, pi-cation pairs, and hydrophobic interactions. Thus, each obligate heterodimer complex comprises two transposase domains. In some embodiments, two fusion proteins provided herein form a complex, said complex comprising (a) a first fusion protein comprising a transposase domain and (b) a second fusion protein comprising a transposase domain; wherein the transposase domains of the first fusion protein and the transpose domains of the second fusion protein have opposing charge that permits the two fusion proteins to form a complex. In non-limiting examples, the assembled complex could be a single dimer (2 protein molecules) or a dimer of dimers (4 protein molecules, or a tetramer).

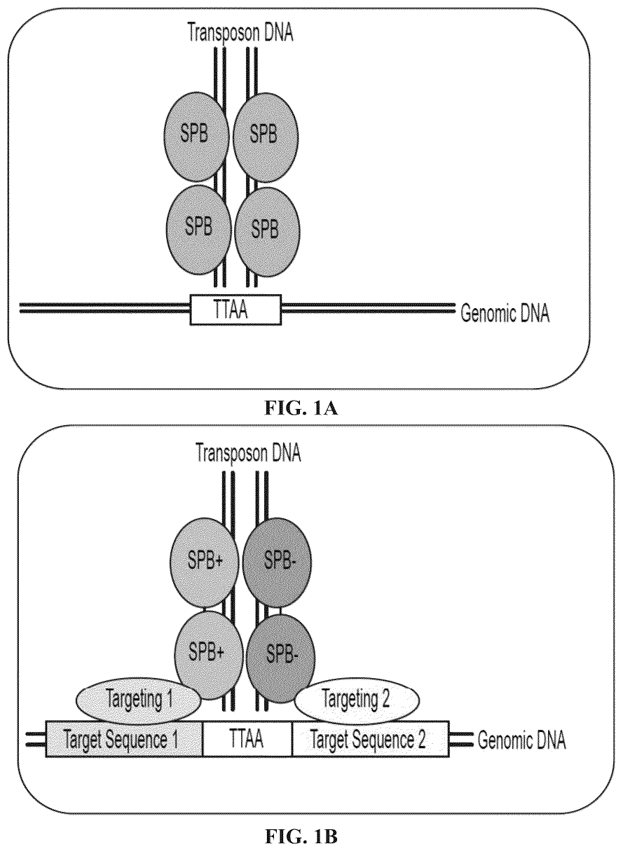

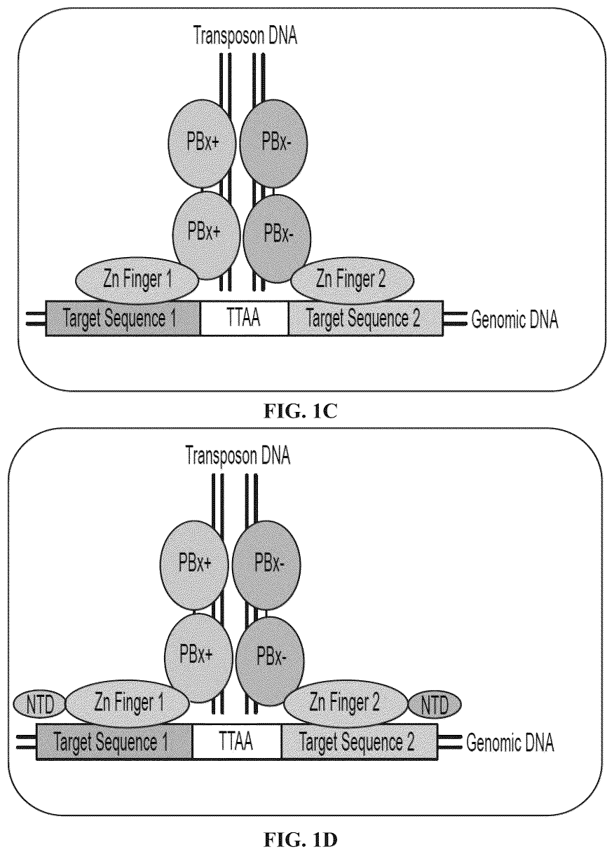

By introducing charged residues into the amino acids that contribute to the dimerization with a second fusion protein, it is possible to design pairs of fusion proteins that can only associate with each other into a tandem dimer in a predetermined configuration. By introducing mutations that only allow for one configuration of the tandem dimer, it becomes feasible to introduce DNA targeting domains into the fusion proteins, thus increasing specificity of the transposase domains. This is illustrated in FIGS. 1A and 1B for SPB and in FIGS. 1C and 1D for PBx: Introducing DNA targeting domains into fusion proteins that can dimerize in any configuration, including homodimerization, would lead to four DNA targeting domains being present in a tandem dimer transposase. However, only two DNA targeting domains would interact with the DNA, leaving the other two to potentially sterically hinder the transposase-DNA interaction. Any suitable DNA targeting domain described herein or known in the art may be used in the fusion proteins described herein.

Mutations in the transposase domains that confer a positive or negative charge can be determined by a person of skill in the art. In the case of a fusion protein comprising a first and second transposase domain, the crystal structure published in Chen et al. (Nat Commun 11, 3446 (2020)) may be used to identify residue pairs in the transposase domains that are in close proximity in the tandem dimer formed by two such fusion proteins. Changing the charge of such residue pairs to create a positively charged transposase domain and a negatively charged transposase domain can be accomplished using standard techniques, such as site-directed mutagenesis.

For example, one or more of M185, R189, K190, D191, H193, M194, D198, D201, 5203, L204, 5205, V207, K500, R504, K575, K576, R583, N586, 1587, D588, M589, C593, and/or F594 may be mutated in an SPB transposase domain (e.g., the SPB set forth in SEQ ID NO: 1 or 2, with numbering beginning at the 12^{th} residue of SEQ ID NO: 1 and at the 5^{th} residue of SEQ ID NO: 2) to generate an SPB− or an SPB+ transposase domain. Similarly, one or more of M185, R189, K190, D191, H193, M194, D198, D201, S203, L204, S205, V207, K500, R504, K575, K576, R583, N586, 1587, D588, M589, C593, and/or F594 may be mutated in a PBx transposase domain (e.g., the PBx transposase domain of SEQ ID NO: 3 with numbering beginning at the 12^{th} residue of SEQ ID NO: 3, or the PBx transposase domain of SEQ ID NO: 4) to generate a PBx− (minus) or a PBx+ (plus) transposase domain.

In some embodiments, a fusion protein described herein may comprise (i) one SPB+ transposase domain, or (ii) one SPB− transposase domain.

To accomplish formation of an obligate heterodimer, pairs of mutations may be introduced into fusion proteins or transposase domains to generate positive and negatively charged fusion proteins or transposase domains which can then interact for form a heterodimer. In some embodiments, the residue pair being mutated is one set forth in Table 2. For example, one or more of the mutations listed in the column labeled “Protein 1” may be introduced into a first SPB or PBx domain and the corresponding mutation or mutations listed in the column labeled “Protein 2” may be introduced into a second SPB or PBx domain. In some embodiments, the members of a residue pair are mutated to have opposing charges.

TABLE 2
Illustrative Residue Pairs; numbering begins at residue
5 of SEQ ID NO: 2 or residue 12 of SEQ ID NO: 1 or 3.

| Protein 1 | Protein 2 | Protein 1 | Protein 2 | Protein 1 | Protein 2 |
|---|---|---|---|---|---|
| M185 | L204 | D201 | R504 | R583 | D588 |
| R189 | R189 | S203 | R504 | N586 | D588 |
| R189 | D191 | L204 | R189 | I587 | R583 |
| R189 | M194 | L204 | L204 | I587 | I587 |
| R189 | L204 | L204 | S205 | D588 | I587 |
| K190 | K190 | L204 | R504 | D588 | D588 |
| K190 | H193 | S205 | L204 | D588 | M589 |
| K190 | M194 | V207 | S203 | M589 | M589 |
| D191 | R189 | V207 | L204 | M589 | F594 |
| H193 | K190 | K500 | D198 | C593 | M589 |
| M194 | R189 | R504 | D201 | F594 | K575 |
| M194 | K190 | K575 | F594 | F594 | K576 |
| D198 | K500 | K576 | F594 | F594 | M589 |

To introduce a positive charge, amino acids with uncharged side chains, such as methionine, or amino acids with a negatively charged side chain, such as aspartic acid, may be changed to positively charged amino acids, such as lysine or arginine. To introduce a negative charge, amino acids with positively charged side chains, such as arginine or lysine, or amino acids with hydrophobic side chains, such as leucine, may be changed to negatively charged amino acids, such as aspartic acid or glutamic acid.

In certain embodiments, one or more of the following mutations is/are introduced into a SPB transposase domain (e.g., the SPB set forth in SEQ ID NO: 1 or 2, with numbering beginning at the 12^{th} residue of SEQ ID NO: 1 and at the 5^{th} residue of SEQ ID NO: 2) of a fusion protein provided herein to generate an SPB+ fusion protein: M185R, M185K, D197K, D197R, D198K, D198R, D201K, and D201R. In some embodiments, an SPB+ transposase domain comprises an M185R mutation and a D198K mutation. In some embodiments, an SPB+ transposase domain comprises an M185R mutation and a D201R mutation. In some embodiments, an SPB+ transposase domain comprises a D197K mutation and a D201R mutation. In some embodiments, an SPB+ transposase domain comprises a D198K mutation and a D201R mutation. In some embodiments, an SPB+ transposase domain comprises an M185R mutation, a D198K mutation, and a D201R mutation.

In certain embodiments, one or more of the following mutations is/are introduced into a PBx transposase domain (e.g., the PBx transposase domain of SEQ ID NO: 3 with numbering beginning at the 12^{th} residue of SEQ ID NO: 3; or the PBx transposase domain of SEQ ID NO: 4) of a fusion protein provided herein to generate an PBx+ fusion protein: M185R, M185K, D197K, D197R, D198K, D198R, D201K, and D201R. In some embodiments, an PBx+ transposase domain comprises an M185R mutation and a D198K mutation. In some embodiments, a PBx+ transposase domain comprises an M185R mutation and a D201R mutation. In some embodiments, an PBx+ transposase domain comprises a D197K mutation and a D201R mutation. In some embodiments, an SPB+ transposase domain comprises a D198K mutation and a D201R mutation. In some embodiments, an PBx+ transposase domain comprises an M185R mutation, a D198K mutation, and a D201R mutation.

In certain embodiments, one or more of the following mutations is/are introduced into a SPB transposase domain (e.g., the SPB set forth in SEQ ID NO: 1 or 2, with numbering beginning at the 12^{th} residue of SEQ ID NO: 1 and at the 5^{th} residue of SEQ ID NO: 2) of a fusion protein provided herein to generate an SPB− fusion protein: L204D, L204E, K500D, K500E, R504E, and R504D. In some embodiments, an SPB− transposase domain comprises an L204E mutation and a K500D mutation. In some embodiments, an SPB− transposase domain comprises an L204E mutation and an R504D mutation. In some embodiments, an SPB-transposase domain comprises a K500 mutation and an R504D mutation. In some embodiments, an SPB− transposase domain comprises an L204E mutation, a K500D mutation, and an R504D mutation.

In certain embodiments, one or more of the following mutations is/are introduced into a PBx transposase (e.g., the PBx transposase domain of SEQ ID NO: 3 with numbering beginning at the 12^{th} residue of SEQ ID NO: 3 or the PBx transposase domain of SEQ ID NO: 4) of a fusion protein provided herein to generate a PBx− fusion protein: L204D, L204E, K500D, K500E, R504E, and R504D. In some embodiments, a PBx− transposase domain comprises an L204E mutation and a K500D mutation. In some embodiments, a PBx− transposase domain comprises an L204E mutation and an R504D mutation. In some embodiments, a PBx− transposase domain comprises a K500 mutation and an R504D mutation. In some embodiments, an PBx− transposase domain comprises an L204E mutation, a K500D mutation, and an R504D mutation.

Illustrative sequences of SPB+ transposase domains are set forth in SEQ ID NOs: 42-54. Illustrative sequences of SPB− transposase domains are set forth in SEQ ID NOs: 55-64. In some embodiments, a transposase domain provided herein comprises the amino acid sequence set forth in any one of SEQ ID NOs: 42-64. In some embodiments, a transposase domain provided herein comprises the amino acid sequence set forth in any one of SEQ ID NOs: 42-64 further comprising one or more conservative amino acid sequences.

In some embodiments, a fusion protein described herein comprises a transposase domain comprising an amino acid sequence set forth in any one of SEQ ID NOs: 42-54. In some embodiments, the transposase domain comprises an amino acid sequence set forth in any one of SEQ ID NOs: 42-54 further comprising one or more conservative amino acid sequences.

In some embodiments, a fusion protein described herein comprises a transposase domain comprising an amino acid sequence set forth in any one of SEQ ID NOs: 55-64. In some embodiments, the transposase domain comprises an amino acid sequence set forth in any one of SEQ ID NOs: 55-64 further comprising one or more conservative amino acid sequences.

In some embodiments, provided herein is a complex comprising (a) a first fusion protein comprising a transposase domain comprising the amino acid sequence set forth in any one of SEQ ID NOs: 42-54; and (b) a second fusion protein comprising a transposase domain comprising the amino acid sequence set forth in any one of SEQ ID NOs: 55-64.

The SPB+, SPB−, PBx+, and PBx− fusion proteins and transposase domains may further comprise the N-terminal deletions of the transposase domain described herein. Thus, in some embodiments, provided herein is an SPB+ fusion protein comprising a transposase domain comprising an N-terminal deletion of about 20 amino acids, about 40 amino acids, about 60 amino acids, about 80 amino acids, about 100 amino acids, or about 115 amino acids. In some embodiments, the transposase domain comprises an N-terminal deletion of 83 amino acids. In some embodiments, the transposase domain comprises an N-terminal deletion of 90 amino acids. In some embodiments, the transposase domain comprises an N-terminal deletion of 84 amino acids. In some embodiments, the transposase domain comprises an N-terminal deletion of 85 amino acids. In some embodiments, the transposase domain comprises an N-terminal deletion of 86 amino acids. In some embodiments, the transposase domain comprises an N-terminal deletion of 87 amino acids. In some embodiments, the transposase domain comprises an N-terminal deletion of 88 amino acids. In some embodiments, the transposase domain comprises an N-terminal deletion of 89 amino acids. In some embodiments, the transposase domain comprises an N-terminal deletion of 90 amino acids. In some embodiments, the transposase domain comprises an N-terminal deletion of 91 amino acids. In some embodiments, the transposase domain comprises an N-terminal deletion of 92 amino acids. In some embodiments, the transposase domain comprises an N-terminal deletion of 93 amino acids. In some embodiments, the transposase domain comprises an N-terminal deletion of 94 amino acids. In some embodiments, the transposase domain comprises an N-terminal deletion of 95 amino acids. In some embodiments, the transposase domain comprises an N-terminal deletion of 96 amino acids. In some embodiments, the transposase domain comprises an N-terminal deletion of 97 amino acids. In some embodiments, the transposase domain comprises an N-terminal deletion of 98 amino acids. In some embodiments, the transposase domain comprises an N-terminal deletion of 99 amino acids. In some embodiments, the transposase domain comprises an N-terminal deletion of 100 amino acids. In some embodiments, the transposase domain comprises an N-terminal deletion of 101 amino acids. In some embodiments, the transposase domain comprises an N-terminal deletion of 102 amino acids. In some embodiments, the transposase domain comprises an N-terminal deletion of 103 amino acids.

In some embodiments, provided herein is an SPB− fusion protein comprising a transposase domain comprising an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the transposase domain comprises an N-terminal deletion of 83 amino acids. In some embodiments, the transposase domain comprises an N-terminal deletion of 84 amino acids. In some embodiments, the transposase domain comprises an N-terminal deletion of 85 amino acids. In some embodiments, the transposase domain comprises an N-terminal deletion of 86 amino acids. In some embodiments, the transposase domain comprises an N-terminal deletion of 87 amino acids. In some embodiments, the transposase domain comprises an N-terminal deletion of 88 amino acids. In some embodiments, the transposase domain comprises an N-terminal deletion of 89 amino acids. In some embodiments, the transposase domain comprises an N-terminal deletion of 90 amino acids. In some embodiments, the transposase domain comprises an N-terminal deletion of 91 amino acids. In some embodiments, the transposase domain comprises an N-terminal deletion of 92 amino acids. In some embodiments, the transposase domain comprises an N-terminal deletion of 93 amino acids. In some embodiments, the transposase domain comprises an N-terminal deletion of 94 amino acids. In some embodiments, the transposase domain comprises an N-terminal deletion of 95 amino acids. In some embodiments, the transposase domain comprises an N-terminal deletion of 96 amino acids. In some embodiments, the transposase domain comprises an N-terminal deletion of 97 amino acids. In some embodiments, the transposase domain comprises an N-terminal deletion of 98 amino acids. In some embodiments, the transposase domain comprises an N-terminal deletion of 99 amino acids. In some embodiments, the transposase domain comprises an N-terminal deletion of 100 amino acids. In some embodiments, the transposase domain comprises an N-terminal deletion of 101 amino acids. In some embodiments, the transposase domain comprises an N-terminal deletion of 102 amino acids. In some embodiments, the transposase domain comprises an N-terminal deletion of 103 amino acids.

In some embodiments, provided herein is a PBx+ fusion protein comprising a transposase domain comprising an N-terminal deletion of about 20 amino acids, about 40 amino acids, about 60 amino acids, about 80 amino acids, about 100 amino acids, or about 115 amino acids. In some embodiments, the transposase domain comprises an N-terminal deletion of 83 amino acids. In some embodiments, the transposase domain comprises an N-terminal deletion of 84 amino acids. In some embodiments, the transposase domain comprises an N-terminal deletion of 85 amino acids. In some embodiments, the transposase domain comprises an N-terminal deletion of 86 amino acids. In some embodiments, the transposase domain comprises an N-terminal deletion of 87 amino acids. In some embodiments, the transposase domain comprises an N-terminal deletion of 88 amino acids. In some embodiments, the transposase domain comprises an N-terminal deletion of 89 amino acids. In some embodiments, the transposase domain comprises an N-terminal deletion of 90 amino acids. In some embodiments, the transposase domain comprises an N-terminal deletion of 91 amino acids. In some embodiments, the transposase domain comprises an N-terminal deletion of 92 amino acids. In some embodiments, the transposase domain comprises an N-terminal deletion of 93 amino acids. In some embodiments, the transposase domain comprises an N-terminal deletion of 94 amino acids. In some embodiments, the transposase domain comprises an N-terminal deletion of 95 amino acids. In some embodiments, the transposase domain comprises an N-terminal deletion of 96 amino acids. In some embodiments, the transposase domain comprises an N-terminal deletion of 97 amino acids. In some embodiments, the transposase domain comprises an N-terminal deletion of 98 amino acids. In some embodiments, the transposase domain comprises an N-terminal deletion of 99 amino acids. In some embodiments, the transposase domain comprises an N-terminal deletion of 100 amino acids. In some embodiments, the transposase domain comprises an N-terminal deletion of 101 amino acids. In some embodiments, the transposase domain comprises an N-terminal deletion of 102 amino acids. In some embodiments, the transposase domain comprises an N-terminal deletion of 103 amino acids.

In some embodiments, provided herein is a PBx− fusion protein comprising a transposase domain comprising an N-terminal deletion of about 20 amino acids, about 40 amino acids, about 60 amino acids, about 80 amino acids, about 81 amino acids, about 82 amino acids, about 83 amino acids, about 84 amino acids, about 85 amino acids, about 86 amino acids, about 87 amino acids, about 88 amino acids, about 89 amino acids, about 90 amino acids, about 91 amino acids, about 92 amino acids, about 93 amino acids, about 94 amino acids, about 95 amino acids, about 96 amino acids, about 97 amino acids, about 98 amino acids, about 99 amino acids, about 100 amino acids, about 101 amino acids, about 102 amino acids, about 103 amino acids, or about 115 amino acids. In some embodiments, the transposase domain comprises an N-terminal deletion of 83 amino acids. In some embodiments, the transposase domain comprises an N-terminal deletion of 84 amino acids. In some embodiments, the transposase domain comprises an N-terminal deletion of 85 amino acids. In some embodiments, the transposase domain comprises an N-terminal deletion of 86 amino acids. In some embodiments, the transposase domain comprises an N-terminal deletion of 87 amino acids. In some embodiments, the transposase domain comprises an N-terminal deletion of 88 amino acids. In some embodiments, the transposase domain comprises an N-terminal deletion of 89 amino acids. In some embodiments, the transposase domain comprises an N-terminal deletion of 90 amino acids. In some embodiments, the transposase domain comprises an N-terminal deletion of 91 amino acids. In some embodiments, the transposase domain comprises an N-terminal deletion of 92 amino acids. In some embodiments, the transposase domain comprises an N-terminal deletion of 93 amino acids. In some embodiments, the transposase domain comprises an N-terminal deletion of 94 amino acids. In some embodiments, the transposase domain comprises an N-terminal deletion of 95 amino acids. In some embodiments, the transposase domain comprises an N-terminal deletion of 96 amino acids. In some embodiments, the transposase domain comprises an N-terminal deletion of 97 amino acids. In some embodiments, the transposase domain comprises an N-terminal deletion of 98 amino acids. In some embodiments, the transposase domain comprises an N-terminal deletion of 99 amino acids. In some embodiments, the transposase domain comprises an N-terminal deletion of 100 amino acids. In some embodiments, the transposase domain comprises an N-terminal deletion of 101 amino acids. In some embodiments, the transposase domain comprises an N-terminal deletion of 102 amino acids. In some embodiments, the transposase domain comprises an N-terminal deletion of 103 amino acids.

### Integration Cassettes

Also provided herein are integration cassettes for site-specific transposition of a DNA molecule into the genome of a cell. In some embodiments, the integration cassette comprises an integration site of the sequence TTAA. In some embodiments, the integration cassette for site-specific transposition of a nucleic acid into the genome of a cell comprises a nucleic acid comprising of or consisting of a central transposon ITR integration site CTTAAA sequence flanked by an upstream TAL array target sequence and a downstream TAL array target sequence, wherein each of the upstream and the downstream TAL array target sequences is separated from the CTTAAA sequence by 12 or 13 base pairs. In some embodiments, each of the at least one upstream and downstream TAL array target site sequences are the same. In some embodiments, each of the at least one upstream and downstream TAL array target site sequences are different each of the at least one upstream and downstream TAL array target site sequences are different. In some embodiments, each of the at least one upstream and downstream TAL Array target sites target a 10 bp sequence of an LPA repeat element.

Also provided are methods for site-specific transposition of DNA molecule into the genome of a cell comprising a stably integrated integration cassette, comprising introducing into the cell: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the CTTAAA sequence of the stably integrated integration cassette.

Also provided are methods for generating an engineered cell by site-specific transposition comprising: introducing into a cell comprising a stably integrated integration cassette: a) a nucleic acid encoding a fusion protein comprising a DNA binding domain and a transposase; wherein the fusion protein is expressed in the cell, and b) a DNA molecule comprising a transposon; wherein the expressed fusion protein integrates the transposon by site-specific transposition into the CTTAAA sequence of the stably integrated integration cassette thereby generating the engineered cell.

### Nucleic Acids

Also provided herein are polynucleotides comprising nucleic acid sequences encoding the fusion proteins described herein. In some embodiments, the polynucleotides are isolated.

The isolated polynucleotides of the disclosure can be made using (a) recombinant methods, (b) synthetic techniques, (c) purification techniques, and/or (d) combinations thereof, as well-known in the art.

Methods of constructing nucleic acids encoding the transposase domains comprising an N-terminal deletion described herein are well known in the art or described herein, for example, PCR-based mutagenesis.

The fusion of the present invention can be generated using any suitable method known in the art or described herein.

The isolated polynucleotides of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library.

Methods of amplification of RNA or DNA are well known in the art and can be used according to the disclosure without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

The polynucleotides of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Expression Vectors and Host Cells

The disclosure also relates to vectors that include polynucleotides of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert may be operatively linked to an appropriate promoter. In some embodiments, the promoter is an EF-1α promoter. The expression constructs will further contain sites for transcription initiation, termination and, in the transcribed region, a ribosome binding site for translation. The coding portion of the mature transcripts expressed by the constructs will preferably include a translation initiating codon at the beginning (e.g., ATG) and a termination codon (e.g., UAA, UGA or UAG) appropriately positioned at the end of the mRNA to be translated, with UAA and UAG preferred for mammalian or eukaryotic cell expression.

Expression vectors may include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotes (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors may include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors may include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Agl4, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Agl4 cells (ATCC Accession Number CRL-1851). In a preferred aspect, the recombinant cell is a P3X63Ab8.653 or an SP2/0-Agl4 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenylation or transcription terminator sequences are typically incorporated into the vector. An example of a terminator sequence is the polyadenylation sequence from the bovine growth hormone gene. In some embodiments, the polyA sequence is an SV40 polyA sequence.

Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The plasmid constructs described herein may be used to deliver nucleic acids encoding the transposase domains or fusion proteins described herein to a cell.

The transposase domains and fusion proteins described herein may also be delivered to a cell using mRNA constructs. Thus, in one embodiment, provided herein is an mRNA sequence encoding a transposase domain or a fusion protein described herein. Such mRNA sequences may be delivered to a cell using a nanoparticle, for example, a lipid nanoparticle. Examples of lipid nanoparticles are described in, e.g., International Patent Applications No. PCT/US2021/055876, No. PCT/US2022/017570, U.S. Provisional Application No. 63/397,268, U.S. Provisional Application No. 63/301,855 and U.S. Provisional Application No. 63/348,614, each of which is incorporated herein by reference in its entirety for examples of lipid nanoparticles that may be used to deliver mRNA constructs encoding the fusion proteins or transposase domains described herein. An mRNA construct may also be delivered to a cell by electroporation or nucleofection. The mRNA may be capped or otherwise modified.

### Cells and Modified Cells

The transposases and fusion proteins described herein may be used in conjunction with a transposon to modify cells. The transposon can be a piggyBac™ (PB) transposon. In some embodiments, when the transposon is a PB transposon, the transposase is a piggyBac™ (PB) transposase a piggyBac-like (PBL) transposase or a Super piggyBac™ (SPB) transposase. Non-limiting examples of PB transposons are described in detail in U.S. Pat. Nos. 6,218,182; 6,962,810; 8,399,643 and PCT Publication No. WO 2010/099296, each of which is incorporated herein by reference in its entirety for examples of transposons that may be used in conjunction with the transposases and fusion proteins described herein. The transposons can comprise a nucleic acid encoding a therapeutic protein or therapeutic agent. Examples of therapeutic proteins include those disclosed in PCT Publications No. WO 2019/173636 and No. WO 2020/051374, each of which is incorporated herein by reference in its entirety for examples therapeutic proteins that may be encoded by a transposon used in conjunction with the transposases and fusion proteins described herein.

Thus, provided herein are modified cells comprising one or more transposon and one or more tandem dimer transposase or fusion proteins described herein. Cells and modified cells of the disclosure can be mammalian cells. Preferably, the cells and modified cells are human cells.

A cell modified using a site-specific transposase fusion protein described herein can be a germline cell or a somatic cell. Cells and modified cells of the disclosure can be immune cells, e.g., lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts. The modified cell can be differentiated, undifferentiated, or immortalized. The modified undifferentiated cell can be a stem cell. The modified undifferentiated cell can be an induced pluripotent stem cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publications No. WO 2019/173636 and WO 2020/051374, each of which is incorporated herein by reference in its entirety.

The methods of the disclosure can modify and/or produce a population of modified T cells, wherein at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2Rβ. The cell-surface markers can comprise one or more of CD45RA, CD95, IL-2Rβ, CCR7, and CD62L.

The disclosure provides methods of expressing a CAR on the surface of a cell. The method comprises (a) obtaining a cell population; (b) contacting the cell population to a composition comprising a CAR or a sequence encoding the CAR, under conditions sufficient to transfer the CAR across a cell membrane of at least one cell in the cell population, thereby generating a modified cell population; (c) culturing the modified cell population under conditions suitable for integration of the sequence encoding the CAR; and (d) expanding and/or selecting at least one cell from the modified cell population that express the CAR on the cell surface. A more detailed description of methods for expressing a CAR on the surface of a cell is disclosed in PCT Publications No. WO 2019/049816 and WO 2020/051374, each of which is incorporated herein by reference in its entirety.

The present disclosure provides a cell or a population of cells wherein the cell comprises a composition comprising (a) an inducible transgene construct, comprising a sequence encoding an inducible promoter and a sequence encoding a transgene, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The disclosure further provides a composition comprising the modified, expanded and selected cell population of the methods described herein.

The modified cells of disclosure (e.g., CAR T-cells) can be further modified to enhance their therapeutic potential. Alternatively, or in addition, the modified cells may be further modified to render them less sensitive to immunologic and/or metabolic checkpoints, for example by blocking and/or diluting specific checkpoint signals delivered to the cells (e.g., checkpoint inhibition) naturally, within the tumor immunosuppressive microenvironment.

The modified cells of disclosure (e.g., CAR T-cells) can be further modified to silence or reduce expression of (i) one or more gene(s) encoding receptor(s) of inhibitory checkpoint signals; (ii) one or more gene(s) encoding intracellular proteins involved in checkpoint signaling; (iii) one or more gene(s) encoding a transcription factor that hinders the efficacy of a therapy; (iv) one or more gene(s) encoding a cell death or cell apoptosis receptor; (v) one or more gene(s) encoding a metabolic sensing protein; (vi) one or more gene(s) encoding proteins that that confer sensitivity to a cancer therapy, including a monoclonal antibody; and/or (vii) one or more gene(s) encoding a growth advantage factor. Non-limiting examples of genes that may be modified to silence or reduce expression or to repress a function thereof include, but are not limited the exemplary inhibitory checkpoint signals, intracellular proteins, transcription factors, cell death or cell apoptosis receptors, metabolic sensing protein, proteins that that confer sensitivity to a cancer therapy and growth advantage factors that are disclosed in PCT Publication No. WO 2019/173636.

The modified cells of disclosure (e.g., CAR T-cells) can be further modified to express a modified/chimeric checkpoint receptor. The modified/chimeric checkpoint receptor can comprise a null receptor, decoy receptor or dominant negative receptor. Examples of null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

The stable chromosomal integration can be a random integration, a site-specific integration, or a biased integration. Without wishing to be bound by theory, it is believed that the addition of DNA binding domains to the tandem dimer transposases described herein improves the site-specificity of the transposases.

The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any gene encoding an immunosuppressive protein, and genes encoding proteins involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

The site-specific transgene integration site can be a non-stable chromosomal insertion. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In an aspect, the transient non-chromosomal insertion of a transgene does not integrate into a chromosome and the modified genetic material is not replicated during cell division.

The site-specific transgene integration site can be a modified binding site for the DNA targeting domain in a transposon domain, fusion protein, or tandem dimer described herein. For example, the TTAA target DNA integration site for SPB may be modified to insert flanking DNA binding sites for the DNA targeting domain comprising three Zinc Finger Motifs (e.g., a DNA targeting domain comprising or consisting of the sequence of SEQ ID NO: 28 or a sequence having at least 75%, at least 80%, at least 85%, at least 90%, at least 95%, at least 98%, or at least 99% identity thereto). For example, it is believed that a DNA targeting domain comprising three Zinc Finger Motifs binds to the DNA sequence GCGTGGGCG. Therefore, the introduction of two copies of the sequence GCGTGGGCG flanking the TTAA target integration site for SPB, is believed to improve site-specific integration of an SPB transposase domain comprising a DNA targeting domain comprising three Zinc Finger Motifs. In some embodiments, the two copies of the sequence GCGTGGGCG are in reverse (5′) and complement (3′) orientation.

In some embodiments, provided herein is a polynucleotide comprising, in 5′ to 3′ order, the reverse complement of the sequence of a target site for a DNA targeting domain, a first spacer, the TTAA target integration site for SPB, a second spacer, and the sequence of target site for a DNA targeting domain. In some embodiments, the first spacer and the second spacer have the same length. In some embodiments, the first and/or the second spacer are 3 bp in length. In some embodiments, the first and/or the second spacer are 4 bp in length. In some embodiments, the first and/or the second spacer are 5 bp in length. In some embodiments, the first and/or the second spacer are 6 bp in length. In some embodiments, the first and/or the second spacer are 7 bp in length. In some embodiments, the first and/or the second spacer are 8 bp in length. In some embodiments, the first and/or the second spacer are 9 bp in length. In some embodiments, the first and/or the second spacer are 10 bp in length.

The modified target site may be introduced into a cell or a cell line to facilitate targeted genomic engineering. For example, a cell line which has been engineered to comprise a modified target site for an SPB or a PBx provided herein can be transfected with said SPB or PBx as well as a transposon comprising donor DNA such that the donor DNA is inserted at the modified target site. In some embodiments, the cell line is a T cell line. In some embodiments, the modified target sequence is introduced into a highly expressed genomic region. In some embodiments, the cell is an in vitro cell, e.g., a cell in cell culture.

For DNA binding domains comprising TALs, the target site is determined by the sequence of the TALs. A person of skill in the art will be able to modify the TAL sequences to achieve the desired target specificity.

The genome modification can be a non-stable chromosomal integration of a transgene. The integrated transgene can become silenced, removed, excised, or further modified.

In some embodiments, the transposase domains, fusion proteins and tandem dimer complexes provided herein have better transposase efficacy than their wildtype equivalents. Transposase activity may be measured by any suitable assay known in the art or described herein, for example, a Split GFP assay. For example, the transposase domains, fusion proteins and tandem dimer complexes provided herein may have comparable on-target genome integration activity to their wildtype counterparts, but have decreased off-target genome integration activity compared to their wildtype counterparts.

In some embodiments, a transposase domain and a DNA targeting domain provided herein has a ratio of on-target to off-target activity that is increased at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the unmodified SPB transposase.

In some embodiments, a transposase domain comprising a DNA targeting domain inserted into the N-terminal region of the transposase domain provided herein has a ratio of on-target to off-target activity that is increased at least 50-fold, at least about 100-fold, at least about 150-fold, at least about 200-fold, at least about 250-fold, at least about 300-fold, at least about 350-fold, at least about 400-fold, at least about 450-fold, at least about 500-fold, at least about 550-fold, at least about 600-fold, at least about 650-fold, at least about 700-fold, at least about 750-fold, at least about 800-fold, at least about 850-fold, at least about 900-fold, at least about 950-fold, or at least about 1000-fold compared to the wildtype transposase domain.

In certain embodiments, the modified cells are used therapeutically in adoptive cell therapy.

Adoptive cell compositions that are “universally” safe for administration to any patient (not just the patient from which they are derived) requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I, Beta-2-Microglobulin (B2M), is reduced or eliminated. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (β2M), and non-naturally occurring polypeptides comprising an HLA class I histocompatibility antigen, alpha chain E (HLA-E) polypeptide is disclosed in PCT Application Publication No. WO 2020/051374, which is incorporated herein by reference in its entirety.

Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD3ζ protein or a portion thereof.

The endodomain can further comprise a cytoplasmic domain. The cytoplasmic domain can be isolated or derived from a third protein. The first protein and the third protein can be identical. The ectodomain can further comprise a signal peptide. The signal peptide can be derived from a fourth protein. The first protein and the fourth protein can be identical. The transmembrane domain can be isolated or derived from a fifth protein. The first protein and the fifth protein can be identical.

The present disclosure also provides a non-naturally occurring chimeric stimulatory receptor (CSR) wherein the ectodomain comprises a modification. The modification can comprise a mutation or a truncation of the amino acid sequence of the activation component or the first protein when compared to a wild type sequence of the activation component or the first protein. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58.

The present disclosure provides a nucleic acid sequence encoding any CSR disclosed herein. The present disclosure also provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

Also provided herein are methods site-specific gene integration. The transposase domains and fusion proteins provided herein may be used to deliver a transgene to a cell and integrate the transgene into a target site. The target site may be, for example, a genomic safe harbor, i.e., a genomic site where a transgene can be integrated in a manner that ensures that the transgene functions predictably and does not cause alterations of the host genomic DNA sequence. In some embodiments, the target site is a repetitive element, such as an LPA sequence. There may be one, two or more target sites within one repetitive element. In some embodiments, the target site is located within an intron (e.g., an intro of the LPA gene).

The site-specific integration may be used in vitro or in vivo. An example of an in vivo application is gene therapy, which involves the delivery of a transgene to the genomic DNA of a cell.

### Formulations, Dosages and Modes of Administration

The present disclosure provides formulations, dosages and methods for administration of the compositions and cells described herein. In one aspect, provided herein is a pharmaceutical composition comprising a tandem dimer transposase or a fusion protein described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Methods of Treatment

In another aspect, provided herein are methods of treating a disease or disorder in a subject, the method comprising administering to the subject a composition comprising the modified cells described herein. The terms “subject” and “patient” are used interchangeably herein. In preferred embodiments, the patient is human.

The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. Non-limiting examples of cancer includes leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In some embodiments, the disease or disorder treated in accordance with the methods described herein is a liver disease or disorder, a urea cycle disorder, a metabolic liver disorder or a hemophilia disease. In some aspects, the metabolic liver disorder can be Ornithine Transcarbamylase (OTC) Deficiency. In some aspects, the metabolic liver disorder can be methylmalonic acidemia (MMA).

In a non-limiting example, the present disclosure provides methods of treating a hemophilia disease in a subject. In some aspects, the hemophilia disease can be hemophilia A. In some aspects, the hemophilia disease can be hemophilia B.

In a non-limiting example, the present disclosure provides methods of treating phenylketonuria (PKU) in a subject.

In some embodiments the present disclosure provides methods of treating an autoimmune disease. In some embodiments, the autoimmune disease is autoimmune neutropenia, Guillain-Barre syndrome, epilepsy, autoimmune encephalitis, Isaacs' syndrome, nevus syndrome, pemphigus vulgaris, deciduous pemphigus, bullous pemphigoid, acquired epidermolysis bullosa, gestational pemphigoid, mucous membrane pemphigoid, antiphospholipid syndrome, autoimmune anemia, myasthenia gravis, autoimmune Graves' disease, thyroid eye disease (TED), Goodpasture syndrome, multiple sclerosis, rheumatoid arthritis, lupus, idiopathic thrombocytopenic purpura (ITP), warm autoimmune hemolytic anemia (WAIHA), chronic inflammatory demyelinating polyneuropathy (CIDP), lupus nephritis, or membranous nephropathy.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2020/051374.

The transposase domains and fusion proteins provided herein may be used to deliver a gene therapy. Gene therapy usually involves the delivery of a transgene to the genomic DNA of a cell. Usually, the transgene replaces a gene that is mutated or otherwise not expressed properly in the cell. For example, the transgene may replace a gene that exhibits decreased, insufficient, and/or altered expression in the cell. In some embodiments, such decreased, insufficient, and/or altered expression may directly or indirectly result in a disease or disorder, such as a liver disease or disorder, a urea cycle disorder, a metabolic liver disorder or a hemophilia disease. The fusion proteins, transposase domains, and complexes described herein may be used to deliver a therapeutic transgene to a cell and integrate the transgene into a target site. In some embodiments, a method of treatment comprises introducing into the cell a fusion protein provided in the present disclosure and a transposon, wherein the transposon comprises, in 5′ to 3′ order: a 5′ITR, the transgene, and a 3′ ITR.

In some embodiments, the therapeutic transgene is a gene that is expressed at lower levels and the lower expression results in a disease or disorder. In some embodiments, the therapeutic transgene is a gene that is expressed in an altered pattern compared to a wildtype gene and the altered expression results in a disease or disorder. Thus, provided herein are methods of treating a disease or disorder caused by or associated with altered gene expression comprising administrating to a subject in need thereof a transposon described herein and a transposase.

The therapeutic transgene delivered to the cell by the fusion proteins, transposase domains, and complexes described herein may encode a therapeutic polypeptide. In some embodiments, the therapeutic polypeptide is Factor VIII polypeptide, Factor IX polypeptide, phenylalanine hydroxylase (PAH), ornithine transcarbamylase (OTC) polypeptide, or methylmalonyl-CoA mutase (MUT1) polypeptide.

In a non-limiting example, the transposase domains and fusion proteins provided herein may be used to deliver a liver directed gene therapy. In some aspects, a liver directed gene therapy can be used to treat Ornithine Transcarbamylase (OTC) Deficiency and the therapeutic polypeptide encoded by the therapeutic transgene can comprise ornithine transcarbamylase (OTC) polypeptide. In some aspects, a liver directed gene therapy can be used to treat methylmalonic acidemia (MMA) and the at least one therapeutic protein encoded by the therapeutic transgene can comprise a methylmalonyl-CoA mutase (MUT1) polypeptide.

In some aspects, a liver directed gene therapy can be used to treat hemophilia A and the at least one therapeutic protein encoded by the therapeutic transgene can comprise Factor VIII. In some aspects, a liver directed gene therapy can be used to treat hemophilia B and the at least one therapeutic protein encoded by the therapeutic transgene can comprise Factor IX.

In some aspects, a liver directed gene therapy can be used to treat phenylketonuria (PKU) and the at least one therapeutic protein encoded by the therapeutic transgene can comprise phenylalanine hydroxylase (PAH).

### Kits

In another aspect, provided herein is a kit comprising a cell line which has been engineered to comprise a modified target site for an SPB or a PBx provided herein within its genome, preferably in a highly expressed genomic region. The kit may further comprise a composition comprising one or more SPB or PBx transposase domains or fusion proteins described herein. In some embodiments, the cell line is a T cell line.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In the context of nucleic acids, a promoter may be operatively linked to a nucleotide sequence encoding a transpose domain or fusion protein described herein, bringing the expression of the nucleotide sequence under the control of the promoter.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 3, Table 4, and Table 5 below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 3.

TABLE 3
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 4.

TABLE 4
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxy1: | S T Y |
| | Amides: | N Q |
| | Sulfhydry1: | C |
| | Borderline: | G Y |

| | |
|---|---|
| Positively Charged (Basic): | K R H |
| Negatively Charged (Acidic): | D E |

Alternately, exemplary conservative substitutions are set out in Table 5.

TABLE 5
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

The Examples in this section are provided for illustration and are not intended to limit the invention.

### Example 1: Construction of Amino-Terminal Deletions of Super PiggyBac Transposases

Plasmids comprising a nucleotide sequence encoding a full-length, wild type Super PiggyBac transposase (SPB; SEQ ID NO: 2) or a nucleotide sequence encoding an integration-deficient variant of Super PiggyBac transposase comprising amino acid substitutions at positions R372A, K375A and D450N (PBx; SEQ ID NO: 3) were used as templates for PCR mutagenesis to generate N-terminal deletion transposase variants lacking the N-terminal 93 amino acids (SPBA1-93 and PBxΔ1-93, respectively).

Briefly, forward and reverse primers were designed to amplify a portion of the SPB and PBx coding sequences corresponding to amino acids 94-594. The resulting DNA fragments encoding SPBΔ1-93 or PBxΔ1-93 were used together with a purchased gBlock gene fragment to construct DNA binding domain-transposase fusion proteins via a state-of-the-art 2-fragment Gibson Assembly.

Additional N-terminal deletion transposase variants lacking the N-terminal 85 amino acids (SPBΔ1-85 and PBxΔ1-85, respectively) were generated as described herein.

### Example 2: Design & Construction of TAL Arrays Targeting LPA

This Example illustrates the design and construction of TAL Array compositions targeting the LPA gene that may be used to in methods to validate the target specificity of TAL Arrays. TAL Arrays were constructed using the design criteria as set forth below.

The Lipoprotein A (LPA) gene contains up to 50 copies of a segmental duplication element making it a potentially attractive target for optimizing the chance of a site-specific transposition event at a target sequence thereby leading to increased number of transposed cells.

TAL Array pairs comprising a N-terminal domain recognizing a T were designed targeting four, specific, 10 bp right and left pair sequences within the repeat elements of the LPA gene. For three of the targets, multiple TAL Array pairs were designed making use of either 12 bp or 13 bp spacers.

The left and right target sequences along with the upstream 5′T used to generate TAL Arrays that target the LPA gene are shown in Table 7.

TABLE 7
Illustrative TAL Arrays Targeting LPA

| LPA PAIR# | LEFT TARGET SEQUENCE | RIGHT TARGET SEQUENCE |
|---|---|---|
| 1 | TGGAGACCCCA (SEQ ID NO: 89) | TCTAGTAATAT (SEQ ID NO: 90) |
| 2 | TGAAGAAACAG (SEQ ID NO: 91) | TTCCTACATGT (SEQ ID NO: 92) |
| | TGAAGAAACAG (SEQ ID NO: 91) | TCCTACATGTC (SEQ ID NO: 93) |
| 3 | TGAAACAAAAT (SEQ ID NO: 94) | TCTTACCTCTA (SEQ ID NO: 95) |
| | TGAAACAAAAT (SEQ ID NO: 94) | TTCTTACCTCT (SEQ ID NO: 96) |
| 4 | TTAAAAAAAAT (SEQ ID NO: 97) | TCCTCTCTGCA (SEQ ID NO: 99) |
| | TTTAAAAAAAA (SEQ ID NO: 98) | TCCTCTCTGCA (SEQ ID NO: 99) |

Individual TAL modules containing 34 amino acid or 20 amino acid “half” repeats were synthesized flanked by BsmBI type IIS restriction sites. The entire module set contains 4 modules capable of recognizing either A, C, G, T for each of 10 bp positions within a target sequence (40 modules/10 bp target). Pairs of TAL arrays targeting sequences in the LPA gene were designed and the corresponding modules were selected and pooled together using “Golden Gate Assembly,” to assemble in frame to create each LPA TAL-Array. All coding sequences used were codon optimized for human expression.

The seven left and right pair combinations were used to design and construct LPA Left TAL Arrays LPAL1, LPAL2, LPAL3, LPAL4.1, and LPAL4.2 (SEQ ID Nos 116, 118, 121, 124, and 125, respectively) and LPA Right TAL Arrays LPAR1, LPAR2.1, LPAR2.2, LPA3.1, LPAR3.2, and LPAR4 (SEQ ID Nos 117, 119, 120, 122, 123, and 126, respectively).

### Example 3: Construction and Analysis of TAL Array-piggyBac Transposase (ss-SPB) Compositions (TAL-PBxs) Designed for Site-specific Transposition at the LPA Gene

This Example illustrates the construction of TAL Array-Super piggyBac transposase fusion protein compositions (TAL-ssSPB) that are useful in methods for achieving site-specific transposition at a specific target locus.

TAL-PBx fusion constructs were prepared as follows: an expression plasmid was synthesized that contains from 5′ to 3′ direction: a CMV promoter, a T7 promoter, a Kozak sequence, a 3× Flag tag (SEQ ID NO: 65), an SV40 NLS (SEQ ID NO: 66), the Delta 152 TAL N-terminal domain (SEQ ID NO: 31), two BsmBI type IIS restriction enzyme sites, the +63 TAL C-terminal domain (SEQ ID NO: 32), a GGGS linker, delta 1-93 PBx (comprising a N-terminal 93 amino acid deletion and mutations at R372A, K375A, D450N in the Super piggyBac transposase codon sequence; SEQ ID NO: 6), and a bGH poly adenylation sequence.

Cloning of a BsmBI-flanked left or right TAL Array into the BsmBI sites of the expression plasmid results in-frame fusion of the TAL Array and the PBx coding sequence via a linker sequence generating full-length TAL-PBx constructs. All coding sequences used were codon optimized for human expression using GeneArt algorithms (Thermo Fisher™).

The eleven TAL Arrays designed and constructed in Example 2 flanked with BsmBI ends were cloned into the BsmBI restriction sites of the expression plasmid described above to generate eleven TAL-PBx constructs: LPAL1, LPAL2, LPAL3, LPAL4.1, and LPAL4.2 Left TAL-PBxs (SEQ ID Nos. 143, 145, 148, 151, and 152 respectively) and LPAR1, LPAR2.1, LPAR2.2, LPA3.1, LPAR3.2, and LPAR4 Right TAL-PBxs (SEQ ID Nos. 144, 146, 147, 149, 150, and 153 respectively).

### Example 4: Demonstration of Site-Specific Transposition Using TAL Array-piggyBac Transposase (ss-SPB) Compositions (TAL-PBxs) and an Episomal Split GFP Splicing Reporter System

This Example illustrates exemplary compositions and methods for demonstrating site-specific transposition at specific episomal loci using TAL Array-SPB transposase fusion proteins.

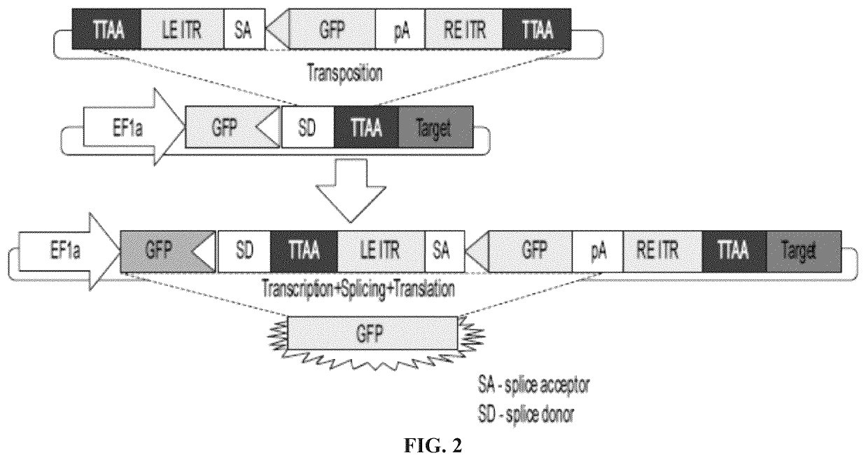

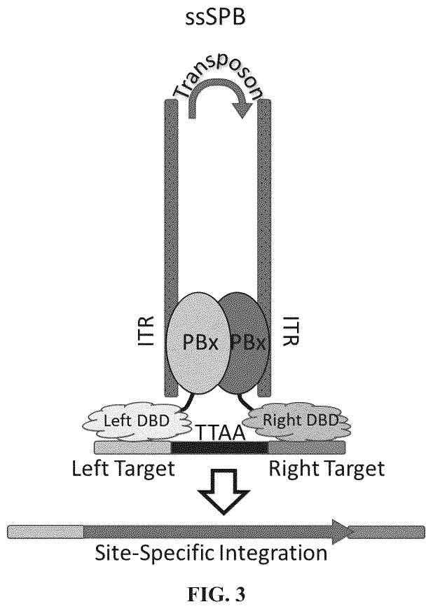

An episomal split GFP splicing reporter system was employed to evaluate site-specific transposition efficiency of the various TAL Array-SPB transposase fusion proteins constructed in Example 3. The reporter system consists of two plasmids. The first plasmid, “the reporter,” was constructed containing from 5′ to 3′ direction: an EF1a promoter (SEQ ID NO: 67), a Kozak sequence, the first portion of a GFP open reading frame (SEQ ID NO: 68), a splice donor (SEQ ID NO: 69), and two BsaI type IIS restriction enzyme sites. The BsaI sites allow for cloning a target TTAA sequence flanked by spacers of variable length flanked by target recognition sequences for TAL arrays. The second plasmid, “the donor,” was constructed containing from 5′ to 3′ direction: a TTAA sequence, the 35 bp PiggyBac minimal 5′ ITR (SEQ ID NO: 70), a splice acceptor site (SEQ ID NO: 71), the second portion of a GFP open reading frame (SEQ ID NO: 72), a synthetic polyadenylation sequence (SEQ ID NO: 73), the 63 bp PiggyBac minimal 3′ ITR (SEQ ID NO: 74), and a TTAA sequence. A schematic of the Split GFP reporter plasmid is shown in FIG. 2.

Four different LPA target sequences naturally found in genomic DNA (SEQ ID Nos. 81-84) were cloned into the episomal reporter plasmid described above. Complementary oligos were synthesized containing the LPA genomic DNA sequences (SEQ ID NOs. 81-84). The complementary oligos contained 4 bp overhangs compatible with the overhangs created in the split GFP splicing reporter following digestion with BsaI. The oligos were annealed and ligated into the digested vector to create a reporter compatible with each LPA TAL-PBx pair constructed in Example 3.

TAL Arrays were designed and constructed to create heterodimeric pairs of TAL-ssSPBs (i.e., one left and one right TAL Array-PBx). Each TAL-PBx construct pair was cotransfected into HEK293T cells with its corresponding reporter plasmid and the donor plasmid. As a negative control, each TAL-PBx construct pair was cotransfected into HEK293T cells with an unmatched reporter plasmid (i.e. TAL-PBx pair 1 with reporter 2, TAL-PBx pair 2 with reporter 3, TAL-PBx pair 3 with reporter 4, and TAL-PBx pair 4 with reporter 1) and the donor plasmid. Transfection mixtures containing 26 ng of the TAL-ssSPB expression vector, 170 ng of the reporter plasmid, 117 ng of donor plasmid and 0.78 ul of Transit-2020 transfection reagent in a total volume of 26 μl of Serum Free OptiMem medium were assembled. 95,000 HEK293T cells in 250 ul of DMEM medium supplemented with 10% FBS were added and the transfection mixture was plated in 48 well plates and incubated for four days at 37° C. at 5% CO2, splitting the cells 1:3 at day two.

When the reporter and donor plasmids are co-transfected into cells along with TAL-PBx, TAL-PBx catalyzes the excision of the transposon from the donor plasmid and its site-specific integration into the TTAA target site of the reporter plasmid. FIG. 3 is a schematic showing the catalytic ssSPB dimer bound to an excised transposon and recognizing its genomic integration target site. Following site-specific transposition, transcription, splicing, and translation, a reconstituted GFP coding sequence is produced (DNA, SEQ ID NO: 75; Amino acid; SEQ ID NO: 76) and fluorescence can be detected. The percentage of on-target site-specific transposition positive cells for the various TAL-PBx pairs were determined by FACS analysis and the results are shown in Table 8.

TABLE 8

| | On-Target | | | Off-Target | | |
|---|---|---|---|---|---|---|
| | Replicate 1 | Replicate 2 | Average | Replicate 1 | Replicate 2 | Average |
| LPA L1/R1 | 21.9 | 21.1 | 21.5 | 4.3 | 3.9 | 4.1 |
| LPA L2/R2.1 | 12.3 | 11.6 | 12.0 | 0.0 | 0.1 | 0.1 |
| LPA L2/R2.2 | 12.9 | 12.5 | 12.7 | 4.8 | 4.5 | 4.7 |
| LPA L3/R3.1 | 16.9 | 15.7 | 16.3 | 0.0 | 0.0 | 0.0 |
| LPA L3/R3.2 | 11.4 | 11.8 | 11.6 | 4.2 | 4.8 | 4.5 |
| LPA L4.1/R4 | 10.4 | 9.6 | 10.0 | 5.0 | 4.6 | 4.8 |

As seen in Table 8, all of the TAL-ssSPB catalyzed site-specific transposition of their respective on-target reporter but not with reporters containing an unmatched off-target. Additionally, the highest transposition was seen at target 1, the only target with a TTTAAA integration site.

### Example 5: Determination of Optimal Flanking 5′ and 3′ Nucleotides Immediately Adjacent to the TTAA Integration Site

The previous Example shows that the target site with the most robust integration, target 1, contains a 5′T and a 3′A immediately adjacent to the TTAA target site, generating a TTTAAA integration site. This Example illustrates additional compositions and methods for preparing optimal target sites for site-specific transposition by determining optimal flanking 5′ and 3′ nucleotides immediately adjacent to the TTAA integration site.

An episomal split GFP splicing reporter as described in Example 4 was employed to evaluate site-specific transposition efficiency of various TAL-PBx fusion proteins targeted to the green fluorescent protein (GFP) gene. TAL Array-SPB transposase fusion proteins GFP1 Right TAL-PBx and GFP1 Left TAL-PBx targeted to specific, 10 bp right and 10 bp left sequences in the coding region of the GFP gene were prepared as described in Examples 14 and 18 of International Patent Application Publication No. PCT/US2022/77549, the contents of which are incorporated by reference in its entirety.

To create a reporter plasmid compatible with the GFP1 Right TAL-PBx, complementary oligos were synthesized containing the target site for the GFP1 Right TAL downstream of a T followed by a 12 bp spacer followed by TTAA followed by a 12 bp spacer, followed by the reverse complement of the TAL target site followed by an A (SEQ ID No. 172). The sequences of the spacers were such that the nucleotide immediately 5′ of TTAA is C and the nucleotide immediately 3′ of TTAA is a C. The complementary oligos contained 4 bp overhangs compatible with the overhangs created in the split GFP splicing reporter following digestion with BsaI. The oligos were annealed and ligated into the digested vector to create a reporter compatible with the GFP1 Right TAL-PBx. Similar oligos were synthesized with 12 bp modified spacers sequences to mutate the flanking 5′ and 3′ nucleotide immediately adjacent to the TTAA integration sequence to a T and an A, respectively, to generate a TTTAAA integration site (SEQ ID No. 173), or to a C and an A, respectively, to generate a CTTAAA integration site (SEQ ID No. 174). Similar oligos were synthesized containing the target site for the GFP1 Right TAL downstream of a T followed by a 13 bp spacer followed by TTAA followed by a 13 bp spacer, followed by the reverse complement of the TAL target site followed by an A (SEQ ID No. 175). The sequences of the spacers were such that the nucleotide immediately 5′ of TTAA is C and the nucleotide immediately 3′ of TTAA is a C. Likewise, similar oligos were synthesized with modified 13 bp spacers sequences to mutate the flanking 5′ and 3′ nucleotide immediately adjacent to the TTAA integration sequence to a T and an A, respectively, to generate a TTTAAA integration site (SEQ ID No. 176), or to a C and an A, respectively, to generate a CTTAAA integration site (SEQ ID No. 177), or to a T and an G, respectively, to generate a TTTAAG integration site (SEQ ID No. 178), or to a C and an G, respectively, to generate a CTTAAG integration site (SEQ ID No. 179).

Each reporter plasmid and donor plasmid were cotransfected into HEK293T cells with the GFP1 Right TAL-PBx expression plasmid (SEQ ID No. 77). As a negative control, the GFP1 Left TAL-PBx expression plasmid (SEQ ID No. 78), which does not recognize the GFP1 Right target sequence, was transfected in place of the GFP1 Right TAL-PBx expression plasmid. HEK293T cells were plated in 24 well plates in 500 μL of DMEM medium supplemented with 10% FBS. The following day, a transfection mixture containing 50 ng of the TAL-ssSPB expression vector, 225 ng of the reporter plasmid, 225 ng of donor plasmid and 1 μL of JetPrime transfection reagent in a total volume of 50 μL of JetPrime buffer were assembled. The mixture was added to the HEK293T cells and the cells were incubated for four days at 37° C. at 5% CO2, splitting the cells 1:6 on day one. The percentage of on-target site-specific transposition positive cells for the various constructs were determined by FACS analysis on day 4.

When the reporter and donor plasmids are co-transfected into cells along with TAL-PBx, TAL-PBx catalyzes the excision of the transposon from the donor plasmid and its site-specific integration into the TTAA target site of the reporter plasmid. Following site-specific transposition, transcription, splicing, and translation, a reconstituted GFP coding sequence is produced (DNA SEQ ID No. 75; Amino acid SEQ ID No. 76) and fluorescence can be detected. The percentage of on-target site-specific transposition positive cells for the various spacer length constructs were determined by FACS analysis and the results are shown in Table 9.

As shown in Table 9, the GFP1 Right TAL-PBx catalyzed site-specific transposition leading to GFP signal above background levels with all target sites. TTTAAA target sites resulted in greater GFP signal than CTTAAC and CTTAAG target sites. CTTAAA and TTTAAG target sites resulted in the greatest GFP signal. GFP1 Left TAL-PBx resulted in no GFP signal above background using the GFP1 Right specific reporters.

TABLE 9

| | % GFP+ Cells | | | | | | | |
|---|---|---|---|---|---|---|---|---|
| | GFP1 Right TAL-ssSPB | | | | GFP1 Left TAL-ssSPB | | | |
| | Replicate | Replicate | Replicate | | Replicate | Replicate | Replicate | |
| | 1 | 2 | 3 | Average | 1 | 2 | 3 | Average |
| 12bp | 15.8 | 15.0 | 15.2 | 15.3 | 3.5 | 3.1 | 3.3 | 3.3 |
| (CTTAAC) | | | | | | | | |
| 12bp | 30.8 | 30.6 | 31.0 | 30.8 | 3.9 | 3.9 | 3.9 | 3.9 |
| (TTTAAA) | | | | | | | | |
| 12bp | 33.4 | 35.5 | 34.8 | 34.6 | 3.5 | 3.6 | 3.4 | 3.5 |
| (CTTAAA) | | | | | | | | |
| 13bp | 20.6 | 23.0 | 19.0 | 20.9 | 4.7 | 5.3 | 4.6 | 4.8 |
| (CTTAAC) | | | | | | | | |
| 13bp | 28.2 | 26.4 | 26.5 | 27.0 | 4.3 | 4.0 | 3.3 | 3.9 |
| (TTTAAA) | | | | | | | | |
| 13bp | 34.9 | 32.6 | 31.0 | 32.8 | 4.1 | 5.1 | 5.8 | 5.0 |
| (CTTAAA) | | | | | | | | |
| 13bp | 33.2 | 32.3 | 31.2 | 32.2 | 4.5 | 4.5 | 5.2 | 4.7 |
| (TTTAAG) | | | | | | | | |
| 13bp | 24.9 | 24.1 | 25.1 | 24.7 | 5.0 | 4.7 | 4.8 | 4.8 |
| (CTTAAG) | | | | | | | | |

### Example 6: Demonstration of Site-Specific Transposition Using TAL Array-piggyflac Transposase (ss-SPB) Compositions (TAL-PBxs)

Based on the results in Example 5, a second set of four different LPA target sequences naturally found in genomic DNA (SEQ TD Nos. 85-88) were cloned into the episomal reporter plasmid described in Example 4. Like the first set of targets evaluated in Example 4, each of the target sequences in the second set have 10 bp TAL binding sites and either 12 bp or 13 bp spacers on both sides of the TTAA. Additionally, each of the target sequences in the second set comprise spacer sequences such that the nucleotide immediately 5′ of TTAA is T and the nucleotide immediately 3′ of TTAA is an A, to generate a TTTAAA integration site, or such that the nucleotide immediately 5′ of TTAA is C and the nucleotide immediately 3′ of TTAA is an A, to generate a CTTAAA integration site. Further, as a thymidine is not immediately 5′ of all the LPA target sites, the TAL N-terminal domain was mutated to not require any specific nucleotide 5′ of the binding site. These mutations were introduced to the wild type TAL sequence by replacing the amino acid sequence QWS at positions 79-81 of SEQ ID NO: 31 with YH to generate the NT-PN variant (SEQ ID NO: 34).

TAL Arrays were constructed to target these TAL binding sites using the design criteria described herein or as set forth below.

TAL Array pairs were designed targeting four, specific, 10 bp right and left pair sequences within the second set of four LPA target sites. For each of the targets, multiple TAL Array pairs were designed making use of either 12 bp or 13 bp spacers.

The left and right target sequences along with the 5′ nucleotide used to generate TAL Arrays that target the LPA gene are shown in Table 10.

TABLE 10
Illustrative TAL Arrays Targeting LPA

| LPA | | |
|---|---|---|
| PAIR# | LEFT TARGET SEQUENCE | RIGHT TARGET SEQUENCE |
| 5 | GTATCCGCAGA (SEQ ID NO: 100) | CGCTTTTCTAC (SEQ ID NO: 101) |
| | TATCCGCAGAG (SEQ ID NO: 102) | GCTTTTCTACA (SEQ ID NO: 103) |
| 6 | AGTATGATAAC (SEQ ID NO: 104) | TCTGCTTTCTT (SEQ ID NO: 105) |
| | GTATGATAACT (SEQ ID NO: 106) | CTGCTTTCTTG (SEQ ID NO: 107) |
| 7 | CGTTTGCTACT (SEQ ID NO: 108) | CTTAATAGATT (SEQ ID NO: 109) |
| | GTTTGCTACTT (SEQ ID NO: 110) | TTAATAGATTA (SEQ ID NO: 111) |
| 8 | GAAGGGAGTGA (SEQ ID NO: 112) | GTACAAGTGTC (SEQ ID NO: 113) |
| | AAGGGAGTGAT (SEQ ID NO: 114) | TACAAGTGTCA (SEQ ID NO: 115) |

The eight left and right pair combinations were used to design and construct LPA Left TAL Arrays LPAL5.1, LPAL5.2, LPAL6.1, LPAL6.2, LPAL7.1, LPAL7.2, LPAL8.1, and LPAL8.2 (SEQ ID Nos 127, 129, 131, 133, 135, 137, 139 and 141, respectively) and LPA Right TAL Arrays LPAR5.1, LPAR5.2, LPAR6.1, LPAR6.2, LPAR7.1, LPAR7.2, LPAR8.1, and LPAR8.2 (SEQ ID Nos 128, 130, 132, 134, 136, 138, 140, and 142, respectively), as described in Example 2.

TAL-PBx fusion constructs were prepared as follows: an expression plasmid was synthesized that contains from 5′ to 3′ direction: a CMV promoter, a T7 promoter, a Kozak sequence, a 3× Flag tag (SEQ ID NO: 65), an SV40 NLS (SEQ ID NO: 66), the Delta 152 TAL N-terminal domain (SEQ ID NO: 31) of the TAL NT-BN variant (SEQ ID NO:34), two BsmBI type IIS restriction enzyme sites, the +73 TAL C-terminal domain (SEQ ID NO: 79), a GGGS linker, delta 1-85 PBx (comprising a N-terminal 85 amino acid deletion and mutations at R372A, K375A, D450N in the Super piggyBac transposase codon sequence; SEQ ID NO: 9), and a bGH poly adenylation sequence.

Cloning of a BsmBI-flanked left or right TAL Array into the BsmBI sites of the expression plasmid results in-frame fusion of the TAL Array and the PBx coding sequence via a linker sequence generating full-length TAL-PBx constructs. All coding sequences used were codon optimized for human expression using GeneArt algorithms (Thermo Fisher™).

The sixteen TAL Arrays flanked with BsmBI ends were cloned into the BsmBI restriction sites of the expression plasmid described above to generate sixteen TAL-PBx constructs: LPAL5.1, LPAL5.2, LPAL6.1, LPAL6.2, LPAL7.1, LPAL7.2, LPAL8.1, and LPAL8.2 Left TAL-PBxs (SEQ ID Nos 154, 156, 158, 160, 162, 164, 166 and 168, respectively) and LPAR5.1, LPAR5.2, LPAR6.1, LPAR6.2, LPAR7.1, LPAR7.2, LPAR8.1, and LPAR8.2 Right TAL-PBxs (SEQ ID Nos 155, 157, 159, 161, 163, 165, 167, and 169, respectively), as described in Example 3.

Additionally, TAL arrays LPAL1 (SEQ ID No 116) and LPAR1 (SEQ ID No. 117) as described in Example 2 were cloned into the expression plasmid described above in this Example 6 to generate TAL-PBx constructs LPAL1 v2 (SEQ ID No 170) and LPAR2 v2 (SEQ ID No 171).

### A. Episomal Target Site-Specific Transposition

The activity of the new mutant TAL-PBx fusions was determined using their respective episomal split GFP splicing reporters. Briefly, each reporter plasmid and the donor plasmid were co-transfected into HEK293T cells with the corresponding TAL-PBx expression plasmid. Approximately 120,000 HEK293T cells were plated in 24 well plates in 500 μl of DMEM medium supplemented with 10% FBS. The following day, a transfection mixture containing 50 ng of the TAL-PBx expression vector, 225 ng of the reporter plasmid, 225 ng of donor plasmid and 1 μl of JetPrime transfection reagent in a total volume of 50 μl of JetPrime buffer were assembled. This mixture was added to the HEK293T cells and they were incubated for four days at 37° C. at 5% CO2, splitting the cells 1:6 at day one. The percentage of GFP positive cells was determined for each sample. The results are shown in Table 11.

TABLE 11

| | On-Target | | | Off-Target | | |
|---|---|---|---|---|---|---|
| | Replicate | Replicate | | Replicate | Replicate | |
| | 1 | 2 | Average | 1 | 2 | Average |
| LPA L1/R1 | 11.2 | 11.5 | 11.4 | 2.0 | 1.8 | 1.9 |
| LPA L1 v2/R1 | 22.6 | 26.2 | 24.4 | 2.0 | 2.1 | 2.1 |
| v2 | | | | | | |
| LPA L5.1/R5.1 | 4.9 | 5.1 | 5.0 | 1.1 | 1.0 | 1.1 |
| (13 bp spacers) | | | | | | |
| LPA L5.2/R5.2 | 4.0 | 4.6 | 4.3 | 1.0 | 1.0 | 1.0 |
| (12 bp spacers | | | | | | |
| LPA L6.1/R6.1 | 19.3 | 22.4 | 20.9 | 1.2 | 1.3 | 1.2 |
| (13 bp spacers) | | | | | | |
| LPA L6.2/R6.2 | 5.9 | 6.5 | 6.2 | 0.8 | 1.1 | 0.9 |
| (12 bp spacers) | | | | | | |
| LPA L7.1/R7.1 | 7.4 | 7.5 | 7.4 | 2.2 | 2.6 | 2.4 |
| (13 bp spacers) | | | | | | |
| LPA L7.2/R7.2 | 3.3 | 3.2 | 3.2 | 1.7 | 2.1 | 1.9 |
| (12 bp spacers) | | | | | | |
| LPA L8.1/R8.2 | 31.5 | 27.9 | 29.7 | 1.9 | 2.1 | 2.0 |
| (13 bp spacers) | | | | | | |
| LPA L8.2/R8.2 | 12.5 | 12.1 | 12.3 | 1.6 | 1.8 | 1.7 |
| (12 bp spacers) | | | | | | |

As seen in Table 11, TAL-ssSPB targeting targets 1, 6, and 8 resulted in the highest transposition. Additionally, TAL-ssSPBs utilizing 13 bp spacers resulted in higher editing than those utilizing 12 bp spacers.

### B. Genomic Target Site-specific Transposition

After confirming the newly designed LPA TALs were functional and recognize their target sequence, the TAL-PBx constructs were used to edit the endogenous genomic LPA targets in Huh7, an immortalized hepatocyte cell line. Briefly, 100,000 cells were plated the day before transfections in 24 well plates in RPMI media+1000 FBS. The following day, 0.5 ug or 1 ug of mRNA encoding LPA Target 1 TAL-ssSPB pair (SEQ TD NOs: 143 and 144) was mixed with 0.5 ul or 1 ul of Messenger Max reagent, respectively, to generate ssSPB-mRNA-lipid complexes. Simultaneously, 450 ng of a transposon donor vector (SEQ TD NO: 80) was mixed with 0.5 ul P3000 reagent and 1 ul of lipofectamine 3000 to generate DNA-lipid complexes. 50 ul of ssSPB mRNA lipid complexes and 50 ul of DNA lipid complexes were delivered to the cells and they were incubated at 37° C.

To assess site-specific integration of the transposon donor into the LPA loci, genomic DNA was extracted from the transfected cells two days post transfections and analyzed by digital droplet PCR (ddPCR) using a probe-based detection scheme. One primer that binds within the transposon was paired with a primer that binds LPA genomic DNA near the TTAA integration site. Therefore, an amplicon should only be generated following site-specific transposition into a LPA locus. Since integration is not directional, two assays were designed for each LPA target to detect integration of the transposon in forward and reverse direction. As a negative control, genomic DNA was extracted from untransfected cells and used as template in the ddPCR reaction to demonstrate the specificity of the primer/probe sets. The results are shown in Table 12.

TABLE 12

| | Percentage of Haploid Genomes Edited at LPA Target | | | | | |
|---|---|---|---|---|---|---|
| | Forward Integration | | | Reverse Integration | | |
| | Replicate 1 | Replicate 2 | Average | Replicate 1 | Replicate 2 | Average |
| Untransfected | 0.00 | 0.00 | 0.00 | 0.00 | 0.00 | 0.00 |
| 0.5 μg mRNA | 0.33 | 0.42 | 0.37 | 0.52 | 0.57 | 0.55 |
| 1 μg mRNA | 0.59 | 0.71 | 0.65 | 1.00 | 1.06 | 1.03 |

As shown in Table 12, amplicons corresponding to forward and/or reverse transposon integration were detected from genomic DNA isolated with cells transfected with LPA TAL-PBx constructs along with the transposon, providing direct evidence of genomic integration at LPA loci.

What is claimed is:

1. A fusion protein comprising a DNA targeting domain and a transposase domain comprising the sequence set forth in SEQ ID NO: 4, wherein the DNA targeting domain binds to a nucleic acid sequence encoding an LPA repeat element.

2. The fusion protein of claim 1, wherein the DNA targeting domain comprises one, two or three Zinc Finger Motifs.

3. The fusion protein of claim 1, wherein the DNA targeting domain comprises one or more TAL domains.

4. The method of claim 3, wherein the TAL domain comprises the sequence set forth in any one of SEQ ID NOs: 35-38.

5. The fusion protein of any one of claims 1-4, wherein the DNA targeting domain binds to a nucleic acid sequence encoding a kringle domain repeat element or an intron adjacent to a sequence encoding a kringle domain repeat element in the LPA gene.

6. The fusion protein of any one of claims 1-5, wherein the transposase domain and the DNA targeting domain are connected by a linker.

7. The fusion protein of claim 6, wherein the linker comprises the sequence GGGGS (SEQ ID NO: 181).

8. The fusion protein of any one of claims 1-7, wherein the DNA targeting domain is inserted into the N-terminus of the transposase domain at a position after the 82^{nd} amino acid and before the 105^{th} amino acid of SEQ ID NO: 4.

9. The fusion protein of any one of claims 1-7, wherein the DNA targeting domain replaces one or more amino acid(s) in the transposase domain between, and including, the 83^{rd} amino acid and the 105^{th} amino acid of SEQ ID NO: 4.

10. The fusion protein of any one of claims 1-9, wherein the transposase domain comprises an N-terminal deletion of amino acids 1-83, 1-84, 1-85, 186, 1-87, 1-88, 1-89, 1-90, 1-91, 1-92, 1-93, 1-94, 1-95, 1-96, 1-97, 1-98, 1-99, 1-100, 1-101, 1-102 or 1-103.

11. The fusion protein of any one of claims 1-10, wherein the transposase domain comprises the sequence set forth in any one of SEQ ID NOs: 7-27.

12. The fusion protein of any one of claims 1-11, wherein the transposase domain comprises (a) at least one mutation selected from the group consisting of M185R, M185K, D197K, D197R, D198K, D198R, D201K, and D201R; or (b) at least one mutation selected from the group consisting of L204D, L204E, K500D, K500E, R504E, and R504D.

13. A polynucleotide comprising a nucleic acid sequence encoding the fusion protein of any one of claims 1-12.

14. A vector comprising the polynucleotide of claim 13.

15. A method of integrating a transgene into a genomic target site of a cell, the method comprising introducing into the cell the fusion protein of any one of claims 1-12 and a transposon, wherein the transposon comprises, in 5′ to 3′ order: a 5′ITR, the transgene, and a 3′ ITR.

16. The method of claim 15, wherein the transposon further comprises an exogenous promoter between the 5′ ITR and the transgene.

17. The method of claim 15 or 16, wherein the transgene encodes a detectable marker.

18. The method of claim 17, wherein the detectable marker is GFP.

19. The method of claim 15 or 16, wherein the transgene is a gene that is (a) not expressed by the cell prior to the introduction of the fusion protein and the transposon or (b) exhibits decreased, insufficient, and/or altered expression by the cell prior to the introduction of the fusion protein and the transposon.

20. The method of any one of claims 15-19, wherein the genomic target site is located on the LPA gene.

21. The method of any one of claims 15-19, wherein the genomic target site is located in a repetitive element.

22. The method of claim 21, wherein the repetitive element is an LPA repeat element.

23. The method of any one of claims 15-19, wherein the genomic target site is located in an intron of a gene.

24. The method of claim 23, wherein the genomic target site is located in the intron of the LPA gene.

25. The method of any one of claims 15-24, wherein the cell is in vivo.

26. A method of modifying the genome of a cell, the method comprising: providing the cell with the fusion protein of any one of claims 1-12, wherein the cell comprises a modified binding site comprising, in 5′ to 3′ order, the sequence of a target site for the DNA targeting domain, a first spacer, a TTAA target integration site for SPB, a second spacer, and the reverse complement of the sequence of the target site for the DNA targeting domain.

27. The method of claim 26, wherein the target integration site comprises the sequence TTAA.

28. The method of claim 26, wherein the target integration site comprises the nucleic acid sequence set forth in any one of SEQ ID NOs: 81-88.

29. An integration cassette for site-specific transposition of a nucleic acid into the genome of a cell comprising a nucleic acid comprising or consisting of a central transposon ITR integration site TTAA sequence flanked by an upstream TAL array target sequence and a downstream TAL array target sequence, wherein each of the upstream and the downstream TAL array target sequences is separated from the TTAA sequence by 12 or 13 base pairs.

30. The integration cassette of claim 29, wherein the integration site comprises the sequence TTAA.

31. The integration cassette of claim 29, wherein the integration site comprises the nucleic acid sequence set forth in any one of SEQ ID NOs: 81-88.

32. The integration cassette of any one of claims 29-31, wherein each of the upstream and downstream TAL array target site sequences are the same.

33. The integration cassette of any one of claims 29-31, wherein each of the upstream and downstream TAL array target site sequences are different.

34. The integration cassette of any one of claims 29-33, wherein each of the upstream and downstream TAL Array target sites target a 7-30 bp sequence of an LPA repeat element.

35. A cell, comprising the integration cassette of any one of claims 29-34 stably integrated into the genome of the cell.

36. A method for site-specific transposition of a DNA molecule into the genome of a cell, comprising introducing into the cell of claim 35:
a) a nucleic acid encoding a fusion protein comprising a DNA binding domain and a transposase; wherein the fusion protein is expressed in the cell; and
b) a DNA molecule comprising a transposon; wherein the expressed fusion protein integrates the transposon by site-specific transposition into the TTAA integration site of the stably integrated integration cassette.

37. A method for generating an engineered cell by site-specific transposition, comprising introducing into the cell of claim 31:
a) a nucleic acid encoding a fusion protein comprising a DNA binding domain and a transposase; wherein the fusion protein is expressed in the cell; and
b) a DNA molecule comprising a transposon; wherein the expressed fusion protein integrates the transposon by site-specific transposition into the TTAA integration site of the stably integrated integration cassette thereby generating the engineered cell.

38. The method of claim 36 or 37, wherein the integration site comprises the sequence TTAA.

39. The method of claim 36 or 37, wherein the integration site comprises the nucleic acid sequence set forth in any one of SEQ ID NOs: 81-88.
